(19) United States
(12) Patent Application Publication
(10) Pub. No.: US 2014/0241987 A1
(43) Pub. Date: Aug. 28, 2014

(54) ALPHA-SYNUCLEIN ANTIBODIES AND USES THEREOF

(71) Applicant: United Arab Emirates University, Al-Ain (AE)

(72) Inventor: Omar EL-AGNAF, Al-Ain (AE)

(73) Assignee: United Arab Emirates University, Al-Ain (AE)

(21) Appl. No.: 13/781,158

(22) Filed: Feb. 28, 2013

(51) Int. Cl. C07K 16/18; A61K 49/00; G01N 33/68

(52) U.S. Cl. CPC C07K 16/18; G01N 33/6854; A61K 49/00
USPC 424/9.1; 530/389.8; 530/388.9; 424/172.1; 435/7.94; 435/7.9

(57) ABSTRACT

The invention describes antibodies having a high affinity for aggregated forms of α-synuclein and a low affinity for monomeric forms of α-synuclein. The antibodies are useful in the diagnosis of neurodegenerative diseases.

### FIELD OF THE INVENTION

The invention relates to the antibodies or fragments thereof that have a high binding affinity for α-synuclein aggregates (e.g. amyloid fibrils, protofibrils or oligomers) and low binding affinity for α-synuclein monomers. The invention is also directed to the use of these antibodies and fragments in the diagnosis, treatment and prevention of neuropathologies associated with α-synuclein.

### BACKGROUND

Synucleins are a family of small proteins, about 14 kDa that are expressed at high levels in nervous tissues. The three members of the family are α-, β-, SNF γ-synucleins.

α-Synuclein is expressed mainly in brain tissues and is primarily located at the presynpatic terminal of neurons. The primary structure of the human form of α-synuclein consists of a 140 amino acid. The wild type sequence of human α-synuclein can be found in FIG. 18. α-Synuclein normally exists as an soluble monomeric protein but can adopt several different folded confirmations depending on its environment. Monomeric α-synuclein can also aggregate into oligomers and into higher molecular weight insoluble fibrils.

Diseases associated with abnormalities in synucleins are often referred to as the synucleinopathies. Synucleinopathies include the neurodegenerative conditions, Parkinson's disease (PD), dementia with Lewy bodies (DLB) and multiple system atrophy (MSA). In synucleinopathies it has been shown that soluble α-synuclein oligomers in brain homogenates of PD and DLB are elevated compared to normal brains. In additional the neuropathologic lesions (Lewy bodies) that often characterise the end stage of PD and DLB have largely been found to be composed of fibrillar α-synuclein deposits.

Commercially available α-synuclein antibodies are known, and are widely used for characterising PD pathology in the brain. However such antibodies are non-specific and recognise both the monomeric and the aggregated forms of α-synuclein, and may not recognise all aggregated forms of α-synuclein.

Known α-synuclein antibodies include Syn-1 (BD Biosciences) and mAb 211 (Santa Cruz Biotechnology), which are known to bind equally to monomeric and aggregated forms of α-synuclein.

WO2011/104696 discloses antibodies that specifically recognise the protofibril forms of α-synuclein. These antibodies exhibit low binding affinity to α-synuclein fibrils and monomers.

### SUMMARY OF THE INVENTION

The invention is directed to antibodies and fragments thereof having a high binding affinity for α-synuclein aggregates and a low binding affinity for monomeric forms of α-synuclein.

The invention is also directed to compositions comprising such an antibody or fragment thereof and to methods of using the antibody or fragment to detect the presence of α-synuclein aggregates and their use in the diagnosis of diseases associated with α-synuclein.

In further embodiments the invention is directed to compositions comprising such an antibody or fragment thereof and methods using the antibody or fragment to treat or prevent diseases associated with α-synuclein pathologies.

Using antibodies specific for α-synuclein aggregates could help reveal novel pathologies for PD and other synucleinopathies.

By producing antibodies specific to the aggregates of α-synuclein and which have low binding affinity to α-synuclein monomers provides new tools to follow the progression of neurodegenerative diseases associated with α-synuclein. The antibodies of the invention also provide antibodies that could be used in the treatment of diseases associated with α-synuclein.

### BRIEF DESCRIPTION OF THE DRAWINGS

The invention will now be described by way of example with reference to the accompanying drawings:

FIG. 1 shows the results of the titer check for the hosts immunized with α-synuclein fibrils (hosts 19, 22, and 23) and α-synuclein oligomers (hosts 210, 211 and 212) as described in Example 1.

FIG. 2 shows the results of the dot plots for screening the specificity of the hybridomas to α-synuclein aggregates, (fibrils (F), oligomers (O) and monomers (M)) as described in Example 2.

FIG. 3 shows the results of the inhibition ELISA using the antibodies Syn-F1, Syn-F2, Syn-O1, Syn-O2, Syn-O3 and Syn-O4 and Syn-1 antibody (BD Bioscience) as a control as described in Example 4.

FIG. 4 shows the results of the cross reactivity of the antibodies Syn-F1, Syn-F2, Syn-O1, Syn-O2, Syn-O3 and Syn-O4 and Syn-1 with amyloid fibrils formed by different proteins as described in Example 5 (A).

FIG. 5 shows the cross reactivity of the antibodies Syn-F1, Syn-F2, Syn-O1, Syn-O2, Syn-O3 and Syn-O4 and Syn-1 with amyloid fibrils formed by β- and γ-synuclein as described in Example 5 (B).

FIG. 6 shows cross reactivity of the antibodies Syn-F1, Syn-F2, Syn-O1, Syn-O2, Syn-O3 and Syn-O4 and Syn-1 with amyloid fibrils formed by α-synuclein peptides NAC (61-98), NAC (61-75) and truncated α-synuclein (1-122) as described in Example 5 (C).

FIG. 7 shows the sensogram results for the mAbs Syn-F1, Syn-F2, Syn-O1, Syn-O2, Syn-O3 and Syn-O4 and mAb211 (Santa Cruz Biotechnology) using Biacore as described in Example 6.

FIG. 8 shows the Pepscan results for the mAbs Syn-F1, Syn-F2, Syn-O1, Syn-O2, Syn-O3 and Syn-O4, and Syn-1 antibody (as a control) by dot blot for epitope determination as described in Example 7.

FIG. 9 shows the results of the Pepscan screening by ELISA as described in Example 7. The plates were probed with the monoclonal antibodies of the invention A) Syn-F1, B) Syn-F2, C) Syn-O1, D) Syn-O2, E) Syn-O3 and F) Syn-O4 or with the control antibodies G) N-19, H) FL-140, I) 5C2, J) 3B6, K) Syn-1 and L) 211.

FIG. 10 shows the plots for the X-ray film placed over the plate of the Pepscan ELISA as described in Example 7. The plot for control antibodies, N-19, FL-140, SC2, Syn-1, Syn-211, 386 is shown in FIG. 10 (A). The antibodies of the invention, Syn-F1, Syn-F2, Syn-O1, Syn-O2, Syn-O3 and Syn-O4 are shown in FIG. 10 (B)

FIG. 11 shows the results of the sandwich ELISA for the detection of α-synuclein aggregates as described in Example 8. The antibodies Syn-F1, Syn-F2, Syn-O1, Syn-O2, Syn-O3 and Syn-O4, and Syn-1 antibody was used as the capturing antibody, FL-140 was used as the detection antibody.

FIG. 12 shows the results of the sandwich ELISA for the detection of phosphorylated Ser129-α-synuclein aggregates as described in Example 9. Antibodies Syn-F1 and Syn-F2, and Syn-1 were used as the capturing antibody and rabbit anti-p-S129-α-synuclein was used as the detection antibody.

FIG. 13 shows the immunhistochemical analysis of tissues from control cases from subjects without Parkinson diseases as described in Example 10.

FIG. 14 shows the immunhistochemical analysis of tissues from Parkinson's disease cases as described in Example 10.

FIG. 15 shows the immunhistochemical analysis of tissues from multiple system atrophy cases as described in Example 10.

FIG. 16 shows the results of Syn-O2 staining in tissues samples from a Parkin carrier PD patient (A), a progressive DLB patient (B) and a classical PD patient as described in Example 11.

FIG. 17 shows the results of staining with the Syn-F2 antibodies of treatments of brain sections from the CA2 region of hippocampus (A), and the Entorhinal cortex neutries (B) as described in Example 12. The section underwent the following treatment: 1) no pre-treatment, 2) autoclaving at 120° C. for 10 minutes in citrate buffer, 3) formic acid for 15 mins or 4) 20 μg/ml proteinase K treatment. A control 5) with the Syn-1 antibody was carried out on tissues samples which had been treated with formic acid for 15 mins.

FIG. 18 shows the wild-type sequence of human α-synuclein.

### DETAILED DESCRIPTION

The invention relates to antibodies having high binding affinity to α-synuclein aggregates and low binding affinity to α-synuclein monomers. The antibodies have an increased affinity to bind to α-synuclein aggregates compared to monomeric α-synuclein forms.

In one embodiment, the invention relates to antibodies or fragments thereof having a high binding affinity for α-synuclein aggregates and low binding affinity for α-synuclein monomers.

Unless otherwise stated the term α-synuclein aggregates is intended to cover early soluble aggregates forms of α-synuclein (such as low and high molecular weight soluble oligomers, including protofibrils) and mature insoluble aggregates forms of α-synuclein (such as mature fibrils). In particular the antibodies and fragments thereof have high binding affinity to α-synuclein fibrils and high binding affinity to α-synuclein oligomers.

Having a high affinity for α-synuclein aggregates means that the antibodies or fragments exhibit a dissociation constant, Kd of less of than 10⁻⁷M for α-synuclein aggregates. Preferably the antibody exhibits a Kd of less than 10⁻⁸M, or less than 10⁻⁹M, or even more preferably a Kd of less than 10⁻¹⁰M, or even less than 10⁻¹¹M. Preferably the α-synuclein is human α-synuclein.

Fibrils are insoluble higher molecular weight aggregated forms of α-synuclein.

Soluble oligomeric forms of α-synuclein come in a variety of sizes and morphologies and includes dimer, trimers, tetramers and multimers. Protofibrils are an intermediate step in the pathway to the formation of the α-synuclein fibrils from the monomeric forms. When the term oligomeric forms of α-synuclein is used this is also intended to include protofibrils.

A fragment thereof of the antibodies means active fragments thereof, i.e. fragments having the same characteristics that are used for the definition of an antibody according to the invention, namely high affinity for α-synuclein aggregates and low binding affinity to α-synuclein monomers. For convenience when the term antibody is used, fragments thereof exhibiting the same characteristic are also being considered.

Having a low binding affinity for α-synuclein monomers means that the binding of an antibody or fragment to α-synuclein monomers is at least 100 times less than the binding to α-synuclein aggregates, preferably about 500 less, or about a 1000 times lower binding affinity to α-synuclein monomers compared to α-synuclein aggregates. In one embodiment the antibody or fragment thereof has a dissociation constant, Kd, of more than 10⁻⁵M for monomeric α-synuclein.

In one embodiment the antibodies may have a higher affinity for α-synuclein fibrils than for oligomeric forms of α-synucleins.

The binding affinities of the antibodies can be determined by using a variety of methods recognised in the art including, surface plasmin resonance based approaches, for example as described in Example 6. Binding can also be evaluated using immunoassays such as ELISA or RIAs. Preferably the binding affinity is determined using surface plasmon resonance assays using a BIACore™ X-100.

In one embodiment the antibodies are conformational antibodies. The antibodies recognise a conformational epitopes, i.e. the epitope the antibody recognises includes the tertiary structure of the aggregates of α-synuclein. In one embodiment the antibodies bind more strongly to the α-synuclein fibrils than to any of the linear peptide epitopes as described in Table 5. In particular the binding of the antibodies is at least 100 times higher to the α-synuclein fibrils than to the linear peptide epitopes as described in Table 5, preferably 500 times more, or preferably 1000 times more.

The antibodies of the invention may bind weakly to a linear epitope within the amino acid region 127-140 of α-synuclein. By binding weakly means that the binding affinity of the antibodies of the invention is at least 100 less to a linear epitope within the amino acid region 127-140 of α-synuclein than the binding affinity of the antibodies to α-synuclein aggregates, in particular at least 100 times less than to α-synuclein fibrils. Preferably the binding affinity to a linear epitope within the amino acid region 127-140 of α-synuclein is 1000 times less than to the binding affinity of the antibodies to α-synuclein aggregates. In one embodiment the antibodies do not recognise or bind to a linear epitope of α-synuclein.

In one embodiment the epitope recognised by the antibody comprises a C-terminal region of α-synuclein. By recognising an epitope comprising a C-terminal region of α-synuclein means that at least part of the epitope the antibody has the ability to bind to includes at least part of the C-terminal region of α-synuclein.

In one embodiment the antibodies of the invention can also bind to aggregated forms of phosphorylated α-synuclein. The antibodies exhibit low binding affinity to monomeric forms of phosphorylated α-synuclein as compared to aggregated forms of phosphorylated α-synuclein, e.g. the binding affinity of the antibodies is at least 100 times less, preferably 500 times less, more preferably 1000 times less to monomeric phosphorylated α-synuclein forms as compared to aggregated phosphorylated α-synuclein. Phosphorylation of the α-synuclein can occur at Ser129.

The antibodies of the invention also exhibit low binding affinity to other amyloid proteins including, β-synuclein, γ-synuclein monomers, IAPP (islet amyloid polypeptide), β-amyloid monomers, Tau and ABri, e.g. the binding affinity of the antibodies is at least 100 times less to one or more of these peptide/proteins than that to the α-synuclein aggregates.

In one embodiment the binding affinity of the α-synuclein aggregate antibodies of the invention is at least 100 times less, preferably 1000 less to β-synuclein than to α-synuclein aggregates. In particular the binding affinity of the antibodies is at least 100 times less, preferably 1000 less to β-synuclein than the binding affinity to α-synuclein fibrils.

In one embodiment the binding affinity of the α-synuclein aggregate antibodies of the invention is at least 100 times less, preferably 1000 less to γ-synuclein than to α-synuclein aggregates. In particular the binding affinities of the antibodies are at least 100 times less, preferably 1000 less to γ-synuclein than to α-synuclein fibrils.

Examples of antibodies according to the invention have been developed by traditional hybridoma techniques. The antibodies may be polyclonal or monoclonal. In a specific embodiment the antibodies are monoclonal.

Typically the antibody is a mammalian antibody, such as primate, human, rodent, rabbit, ovine, porcine or equine antibody. The antibody can be any class or isotype antibody, for example IgM or IgG. Preferably the antibody is IgG.

In another aspect of the invention the antibodies can be used as diagnostic tools for detecting the presence of α-synuclein aggregates in a sample. The antibodies may be used for monitoring and/or diagnosing neurodegenerative disorder associated with α-synuclein in an individual.

These antibodies may be suitable as diagnostic tools for neurodegenerative disorders associated with α-synuclein, including but not limited to Parkinson's disease, dementia with Lewey bodies and other α-synuclein related neurodegenerative disorders.

In one embodiment the invention relates to a method of detecting α-synuclein aggregates comprising the steps of:
adding the antibody or fragment thereof to a biological sample and
detecting the presence of a complex formed between α-synuclein aggregates and the antibody or fragment.

The method can detect fibrils and oligomers of α-synuclein.

The method can be carried out in vitro on a tissue or biological fluid sample. The sample obtained from the individual to be tested, can for example be cerebrospinal fluid, (CSF), blood, urine, saliva, or brain, gut, colon, skin or salivary gland tissues. In particularly preferred methods the sample is a CSF sample. In another preferred method the sample is a brain tissue sample.

The sample is combined with the antibody for a time and under conditions effective to allow binding of the antibody to α-synuclein aggregates in the sample.

The sample may be processed prior to being assayed using standards methods. In one embodiment the tissue sample under goes no pre-treatment before testing with the antibody. By pre-treatment it is meant the tissue sample obtained is not subjected to any treatment such as, autoclaving, formic acid and/or proteinase K treatment.

Standard methods known in the art may be used to detect and/or measure the level of the complex formed between the antibodies and α-synuclein aggregates in the sample.

Analysis for the presence of α-synuclein can be conducted from a method such as radioimmunoassay, an enzyme-linked immunosorbant assay (ELISA), a sandwich immunoassay, a fluorescent immunoassay, a precipitation reaction, a gel immunodiffusion assay, an agglutination assay, a protein A immunoassay, an immunoelectrophoresis assay, an electrophoresis, western blotting. Other suitable techniques able to measure and/or detect the presence of α-synuclein in the sample to be tested can also be used.

In one embodiment the antibodies may be coated onto a surface, such as a microwell plate or diagnostic test strip, and the sample added to the antibody and allowed to combined under conditions effective to allow binding. The presence of the complex can then be detected.

In a preferred method an ELISA assay is used to detect and/or quantify the amount of α-synuclein aggregates. In one embodiment the invention is directed to a sandwich ELISA comprising adding the sample to be tested to a microplate, where the surface of the microplate has been coated with an antibody according to the invention; allowing any α-synuclein aggregates present in the sample to bind to the antibodies; and detecting the presence of any antibody/α-synuclein aggregate complexes. Detection can be carried out using labelled antibodies that bind α-synuclein.

The methods can be used for the diagnosis of neurodegenerative diseases and/or monitoring the progression of a neurodegenerative disease. The amount and/or size of any α-synuclein aggregates can be detected.

The neurodegenerative disease can include but is not limited to Parkinson's disease, dementia, Alzheimer's disease, Down's syndrome, multiple-system atrophy, psychosis, schizophrenia or Creutzfeldt-Jakob disease. The dementia may be dementia with Lewy bodies.

The invention also relates to a method of diagnosing a neurodegenerative disease associated with α-synuclein. The method comprises administering an antibody of the invention to an individual and detecting the presence or absence of α-synuclein aggregates. The presence or absence of a complex formed between the α-synuclein aggregates and the antibody can be detected.

The presence of α-synuclein aggregates indicates that the individual has a neurodegenerative disease and the absence of α-synuclein aggregates indicated that the subject does not have the neurodegenerative disease.

An increased level of the complex compared to a base line level will typically indicate that the individual has or is in the process of developing an α-synuclein pathology. The base line level will typically be calculated from a sample from an individual known not to have an α-synuclein pathology (a “normal individual”) or from an earlier test of a sample taken from the same individual being tested.

A correlation has been shown to exist between CSF α-synuclein oligomers levels and disease severity. Detecting the presence and/or amount of the oligomers or fibrils in the sample can be used to follow the progression and or severity of a neurodegenerative disease, in particular for using the antibodies as biomarker in Parkinson diseases and other diseases associated with α-synuclein pathologies.

In one embodiment of the invention the antibodies are used to diagnosis whether an individual has Parkinson's disease. A CSF sample is taken from the patient. Antibodies are contacted with the sample in conditions effective to allow complexes to form between the antibodies and aggregated α-synuclein present in the sample. The presence of the antibody complexes can then be detected.

In one embodiment of the invention the antibodies can be used in an ELISA to measure aggregated α-synuclein in CFS. The antibodies can be used to measure aggregated α-synuclein in a sample with high sensitivity and specificity compared to ELISA using other antibodies. In particular an ELISA using the antibodies of the invention has a higher sensitivity and specificity to detect α-synuclein oligomers and protofibrils in CSF as compared to an ELISA using mAb 211 as a capture antibody and biotinylated 211 as a detection antibody.

The methods can be used to monitor the effectiveness of a therapeutic agent, by using the results of the analysis undertaken. An effective therapeutic agent can be determined as one that causes a decrease in the amount of α-synuclein aggregates present in a sample taken, as compared to a reference value. The reference value may reflect the amount of α-synuclein in the patient before treatment, or may representative a typical amount of α-synuclein to be found in untreated patients.

The antibodies may be labelled with a detectable label. The label will be one that allows detection of the antibody when bound to the α-synuclein aggregates. Detectable labels include, but are not limited to fluorescent labels, radioactive labels and contrast agents. Suitable radiolabels include those such as F¹⁸, I¹²³, In¹¹¹, I¹³¹, C¹⁴, H³, Tc^{99m}, P³², I¹²⁵ and Gallium 68. Suitable fluorescent labels can include fluorescein and rhodamine. Suitable contrast agents include: rare earth ions such as gadolinium (Gd), dysprosium and iron, and magnetic agents. Other labels include nuclear magnetic resonance active labels, positron emitting isotopes detectable by a PET scanner, chemiluminescent and enzymatic markers.

The antibodies can be labelled by standard techniques.

In another aspect of the invention the antibodies can be used as an imaging agent. In particularly the antibodies can be used for detecting and localization and/or quantitation of α-synuclein aggregates in human and animal tissues.

The invention provides a method of imaging α-synuclein aggregates, comprising detecting the binding of the antibody to α-synuclein aggregates.

In one embodiment antibodies of the invention can be contacted with a sample and then the antibody in the sample that has bound to α-synuclein aggregates can be detected. The antibody is preferably a labelled antibody. The presence or absence of the α-synuclein aggregates may be detected in the brain in vivo using any suitable imaging techniques. In such in vivo methods, the method may further comprise administering the antibody to an individual and detecting the antibody.

Suitable imaging techniques include positron emission tomography (PET), gamma-scintigraphy, magnetic resonance imaging (MRI), functional magnetic resonance imaging (FMRI), magnetoencephalography (MEG), and single photon emission computerized tomography (SPECT).

The presence or absence of α-synuclein aggregates may also be detected in vitro, for example in tissue samples, such as a brain section. In such embodiments suitable imaging techniques may also include electron microscopy, confocal microscopy or light microscopy.

The number and/or size of α-synuclein aggregates present in the brain of an individual correlates with the progression of the α-synuclein associated disease. An increase in the size or number of α-synuclein aggregates indicates a progression of the disease, whilst a decrease in the size or number of α-synuclein aggregates indicates a regression of the disease.

The diagnostic methods can also be for use in veterinary use.

The invention also relates to a kit comprising an antibody according to the invention for carrying out the diagnostic methods. The antibody may be an intact immunoglobulin molecule or fragment thereof such as Fab, F(ab)2 or Fv fragment. The antibody may be labelled as described above. The kit can be for use in a method of determining whether an individual has an neurodegenerative disease.

The kit may additionally comprise one or more other reagents or instruments which enable any of the methods to be carried out. Such reagents or instruments including, but not limited to one or more of the following, suitable buffers, means to obtain a sample from an individual, a support comprising wells on which quantitative reactions can be done. The kit may optionally comprise instructions for carrying out the methods above.

In one embodiment of the invention the antibody and fragment thereof can be used as a medicament.

The invention relates to antibodies or fragments thereof for use in the treatment of a neurodegenerative disorder associated with α-synuclein in an individual.

The invention also relates to a method of treating a neurodegenerative disorder with α-synuclein pathology in an individual, comprising administering to the individual a therapeutically effective amount of the antibody or fragment thereof.

The neurodegenerative disorder can include but is not limited to Parkinson's disease, dementia, Alzheimer's disease, Down's syndrome, multiple-system atrophy, psychosis, schizophrenia or Creutzfeldt-Jakob disease. The dementia may be dementia with Lewy bodies.

α-Synuclein aggregation may be reduced or inhibited by the administration of an antibody or fragment thereof. The antibody may be administered to a sample comprising soluble synuclein species or directly to an individual.

An individual may be a human or non-human animal. The compositions and methods as herein described can also be used in veterinary practice.

The terms ‘treatment’ and “treating” and the like, is intended to include curing, relieving, reversing, alleviating, managing or delaying the onset, of the condition, or to reduce the risk of developing or worsening the condition. The terms are also intended to include palliative, prophylactic and preventative treatment of the condition.

In one embodiment of the invention a pharmaceutical composition comprises the antibody or fragment thereof and a pharmaceutically acceptable diluent or carrier.

The antibody may be administered directly to the site of α-synuclein aggregate deposit, e.g. a Lewy body, typically by injection into a blood vessel supplying the brain or into the brain itself.

In general, the nature of the carrier will depend on the particular mode of administration being employed. Pharmaceutical forms include solid, solutions and suspensions. Suitable pharmaceutical carriers include inert diluents or fillers, water and various organic solvents. Compositions may also include additional ingredients such as flavourings, binders & excipients.

Forms suitable for oral administration include tablets, capsules, pills, powders, sustained release formulations, solutions, and suspensions. Forms suitable for parental injection include sterile solutions, suspensions or emulsions.

Exemplary parenteral administration forms include suspensions or solutions in sterile aqueous solutions, for example aqueous propylene glycol or dextrose solutions. Such dosage forms can be suitably buffered, if desired.

Exemplary oral forms such as tablets may include: disintegrates such as starch, alginic acid and complex silicates; binding agents such as sucrose, gelatin and acacia; and lubricating agents such as magnesium stearate, sodium lauryl sulfate and talc. Solid compositions may also include soft and hard gelatin capsules. Preferred materials include lactose, milk sugar and high molecular weight polyethylene glycols.

Methods of preparing various pharmaceutical compositions are well known to those skilled in the art. Reference is made to ‘Remington's Pharmaceutical Sciences’.

The invention also relates to the antibody and fragment in combination with one or more further therapeutic agents.

### EXAMPLES

The following examples are provided for illustration and are not intended to limit the invention to these specific examples.

### Example 1

### Preparation α-Synuclein Aggregates Antibodies

### Preparation of α-Synuclein Fibrils

Purified recombinant α-synuclein was used. Freshly prepared α-synuclein (50 μM) solution was incubated at 37° C. in a thermomixer (800 rpm) for 7 days for aggregation. The aggregation process of α-synuclein was monitored by Thioflavin-S (Th-S) binding assay. Once the aggregation was complete, the fibrils were aliquoted into small samples and stored at −80° C. until used.

### Preparation of α-Synuclein Oligomers

Freshly prepared α-synuclein solution was mixed with dopamine at 1:7 molar ratio (α-synuclein:dopamine) and incubated at 37° C. in a thermomixer (800 rpm) overnight. Next day the solution containing oligomers was aliquoted into small samples and stored at −80° C. until used.

### Immunization

Balb/c female mice were used for sub-cutaneous immunization with α-synuclein. Each mouse received an initial immunization of 50 μg of the α-synuclein solution mixed with Freunds Complete Adjuvant (1:1 v/v) followed by booster immunization, 3 weeks interval, with 25 μg of α-synuclein mixed with Freunds Incomplete Adjuvant (1:1 v/v). On the 10^{th} day of each booster immunization, blood was collected from tail vein and serum was separated. ELISA was carried out to check the immune response of the hosts to α-synuclein.

### ELISA to Check the Immune Response of the Hosts.

A 96 well clear maxisorp plate (NUNC) was coated with 100 μl per well (70 ng/well) for overnight incubation in PBS. The plate was then washed three times with PBST (PBS containing 0.05% Tween-20), and then incubated with blocking buffer (PBST containing 2.25% gelatin) for 1 h at room temperature. The plate was washed three times with PBST and 100 μl of serially diluted antiserum (1/100 followed by 10 dilutions) form the mice were added to the wells in duplicates and the plate was incubated at room temperature for 1 h. After the plate was washed PBST, 1:20000 diluted goat anti-mouse IgG-HRP (100 μl per well, Jackson Immunoresearch) was added to each well and incubated for 1 h at room temperature. The plate was then washed three times with PBST and incubated with 100 μl of TMB substrate (KPL, Gaitherburg, USA) until colour developed. The reaction was stopped by adding H₂SO₄ (0.6 N, 100 μl, per well), and absorbance values at 450 nm were measured using a Victor X3 microtiter plate reader. The hosts that showed good immune response were then taken for fusion.

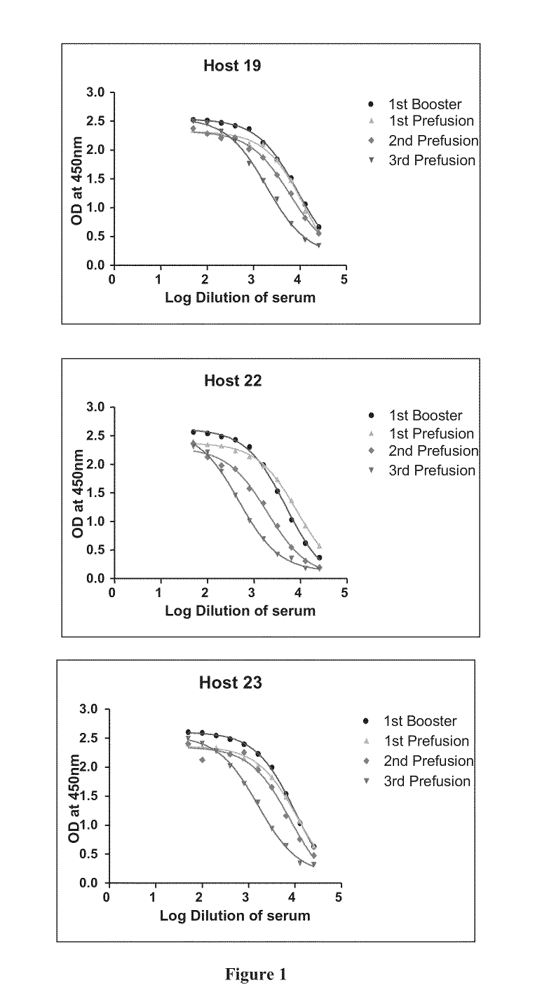

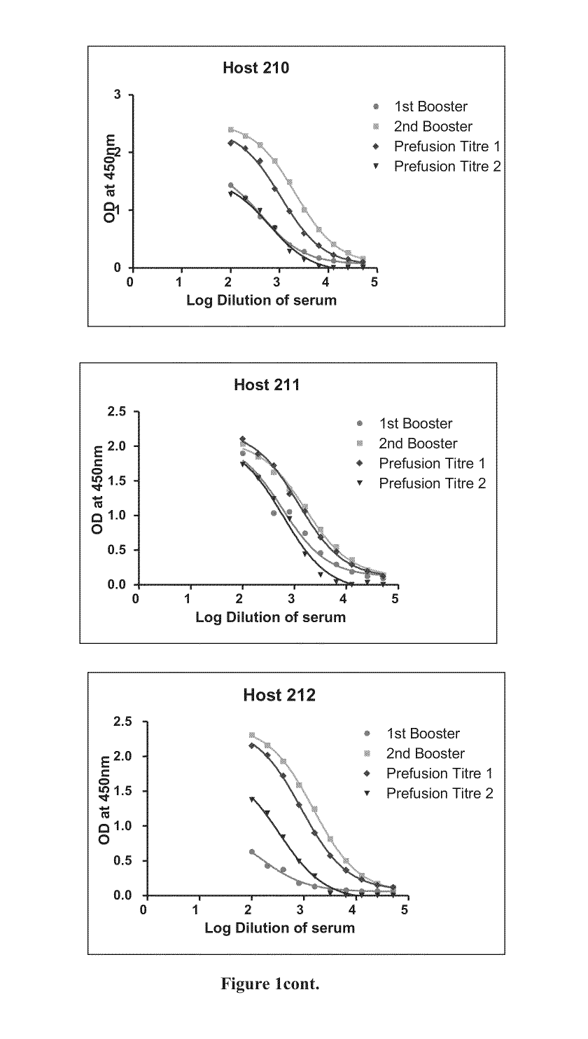

Titer values for the hosts immunized with α-synuclein fibrils (hosts 19, 22, and 23) and α-synuclein oligomers (hosts 210, 211 and 212) were taken and the results are shown in FIG. 1 and Table 1. The hosts were taken for fusion when the titer values came down to half of the previous response.

TABLE 1

| | | 1^{st} Booster | 1^{st} Pre- | 2^{nd} Pre- | 3^{rd} Pre- |
|---|---|---|---|---|---|
| Immunogen | Host ID | titer | fusion titer | fusion titer | fusion titer |
| α-synuclein | 19 | 8871 | 13461 | 6061 | 1923 |
| fibrils | 22 | 4924 | 8543 | 1939 | 475 |
| | 23 | 10514 | 11402 | 7905 | 1595 |
| α-synuclein | 210 | 430 | 2138 | 1059 | 614 |
| oligomers | 211 | 578 | 1608 | 1205 | 604 |
| | 212 | 155 | 1522 | 891 | 339 |

Mice immunized with α-synuclein fibrils showed a very high titer immediately after the first booster. Hence no further booster immunization was given to these hosts. Mice immunized with α-synuclein oligomers showed a satisfactory response after the second booster. Before taking the mice for fusion, pre-fusion titer (after 1 month rest) was carried out. Some hosts showed a high titer even after 2 months of rest owing to the high immunogenicity of the α-synuclein aggregates.

Fusion of Splenocytes with Sp2/O Myeloma Cells

The mice of the previous experiments were given a final intra-peritoneal immunization, 3 times the initial dose, in PBS. After 3 days, the mouse was sacrificed. The spleen was isolated aseptically and washed in IMDM (Gibco). Splenocytes were extracted, counted and fused with Sp2/0 myeloma cells using 50% PEG 4000 (Merck). The fused cells were re-suspended in IMDM growth media (IMDM containing 2 mM glutamax (Gibco), 1× Penstrep (100 U/ml penicillin and 100 μg/ ml streptomycin; Sigma), 50 μg/ml Gentamycin (Sigma), 50 μM 2-mercaptoethanol (Sigma) and 20% fetal bovine serum, Hyclone) supplemented with HAT (1×, Sigma) and macrophages, 6×10⁵ cells/plate (freshly isolated from 5-6 weeks old Balb/c mice). The cells were plated (200 μl/well) into 96-well tissue culture plates (Nunc) and grown at 37° C., 5% CO₂. After one week of incubation, the media form the plates were changed to freshly prepared IMDM growth media supplemented with HT (1×, Sigma). The hybridomas were allowed to grow until the media colour changes to yellow. The culture supernatant was used for screening of positive clones.

### Screening for Positive Clones

A 96 well clear maxisorp plate (Nunc) was coated by overnight incubation with 100 μl per well (70 ng/well) of either α-synuclein fibrils or oligomers in PBS. The plate was then washed three times with PBST (PBS containing 0.05% Tween-20), and incubated with the blocking buffer (PBST containing 0.05% Tween 20) for 1 h at room temperature. Then the plate was washed with PBST before adding 100 μl/well of culture supernatant from the fusion plate. The plate was incubated at room temperature for 1 h. After the plate was washed with PBST (3×), 1:20000 diluted goat anti-mouse IgG-HRP (100 μl per well, Jackson Immunoresearch) was added to each well and incubated for 1 h at room temperature. The plate was then washed with PBST and incubated with 100 μl of TMB substrate (KPL, Gaitherburg, USA) until colour developed. The reaction was stopped by adding 100 μl H₂SO₄ (0.6 N), and the absorbance at 450 nm was measured using a Victor³ 1420 multilabel microtiter plate reader. The positive clones were transferred to a 24 well plate and were screened again to identify the stable clones.

A total of 1100 positive clones were obtained from the initial screening and 57 positive clones (from mouse immunized with α-synuclein fibrils), and 43 clones (from mice immunized with α-synuclein oligomers) were selected for further characterization. The isotype of the positive clones were identified using isotyping kit (Sigma). IgG clones were selected, passaged multiple times to identify stable clones and taken for single cell cloning.

### Isotyping

A 96 well clear maxisorp plate (Nunc) was coated by overnight incubation at 4° C. with 100 μl/well well of 1/1000 diluted anti-mouse heavy chain antibodies (Sigma, Isotyping Kit) in PBS, pH 7.4. The plate was then washed 3 times with PBST (PBS containing 0.05% Tween 20), and incubated with blocking buffer (PBS containing 2.5% gelatin and 0.05% Tween 20; 400 μl per well) for 1 h at room temperature. The plate was then washed 3 times with PBST, and 100 μl of culture supernatant from each clone was added to the wells. The plate was incubated at room temperature for 1 h, and after the plate was washed with PBST, 1:20000 diluted goat anti-mouse IgG-HRP (100 μl/well) was added to each well and incubated for 1 h at room temperature. The plate was then washed with PBST and incubated with 100 μl/well of TMB substrate (KPL, Gaitherburg, USA) until the colour developed. The reaction was stopped by adding 100 μl/well of H₂SO₄ (0.6 N), and the absorbance at 450 nm were measured by Victor³ 1420 multilabel microtiter plate reader. The IgG positive clones were transferred to a T-25 flask and taken for single cell cloning.

Isotypes of clones obtained from mice immunized with α-synuclein fibrils or oligomers is shown in Table 2.

TABLE 2

| | |
|---|---|
| Clones obtained from | Clones obtained from |
| mice immunized with | mice immunized with - |
| α-synuclein fibrils | synuclein oligomers |

| Clone Name | Isotype | Clone Name | Isotype |
|---|---|---|---|
| Syn-F1 | IgG1 | Syn-O1 | IgG2b |
| Syn-F2 | IgG2a | Syn-O2 | IgG1 |
| Syn-F3 | IgG2a | Syn-O3 | IgG1 |
| Syn-F4 | IgG1 | Syn-O4 | IgG1 |
| Syn-F5 | IgG1 | Syn-O5 | IgG1 |
| Syn-F6 | IgG2b | Syn-O6 | IgG1 |
| | | Syn-O7 | IgG1 |
| | | Syn-O8 | IgG1 |

From 100 clones identified and selected, 33 parental clones were found to be IgG isotype. Remaining clones were either IgM or a mixture of IgG and IgM isotypes. The IgM or mixed isotype clones were frozen and not taken for further characterization. The IgG clones were passaged at least 3 times and 14 stable clones were selected. 10 clones were found to be IgG1, 2 clones were IgG2a and 2 were found to be of IgG2b isotype.

### Single Cell Cloning

Cells were collected from T-25 flask of positive hybridoma clones. The cells were counted, diluted in IMDM and ˜100 cells were taken and added to 20 ml of growth media. Growth medium was IMDM (Gibco) supplemented with glutamax (2 mM, Gibco), Penstrep (Sigma), 50 μl g/ml Gentamycin (Sigma), 50 μM beta-ME (Sigma), 20% fetal bovine serum (Hyclone) and macrophages (6000 cells/well) freshly isolated from young BALB/c. The cells were mixed and plated (200 μl/well) in a 96 well tissue culture plate. The plate was incubated at 37° C., 5% CO₂. The hybridomas were allowed to grow, wells containing single cells were marked, until they grew confluent. The culture supernatant from the wells showing single colony was taken for screening by ELISA and the cells from positive wells were transferred to 24 well plates, screened again before transferring to T-25 flask. The culture supernatant from T-25 flask was screened at least thrice to select stable clones.

Single cell clones obtained from each clones were transferred to 24 well plate, and further checked for stability. At least two single cell clones from each parental clone were transferred to T-25 flask for further characterization.

### Example 2

### Screening the Specificity of Hybridoma to α-Synuclein Aggregates by Dot Blot

50 ng (5 μl in PBS) of α-synuclein fibrils (F), oligomers (O) or monomers (M) were spotted onto a nitrocellulose membrane and dried at room temperature for 30 minutes. The membranes were blocked with 5% skimmed milk in PBST (PBS containing 0.05% Tween 20) for 1 h at room temperature. The membranes were then washed 3 times with PBST before probing with culture supernatant 1:1 diluted in PBST and incubated for 2 h at room temperature. The Syn-1 antibody was used as control. After the membranes were washed with PBST, 1:20000 diluted goat anti-mouse IgG-HRP was added and incubated for 1 h at room temperature. The membranes were then washed with PBST followed by 2 times with PBS. The membranes were developed with Super signal West pico Chemiluminescent substrate (Pierce).

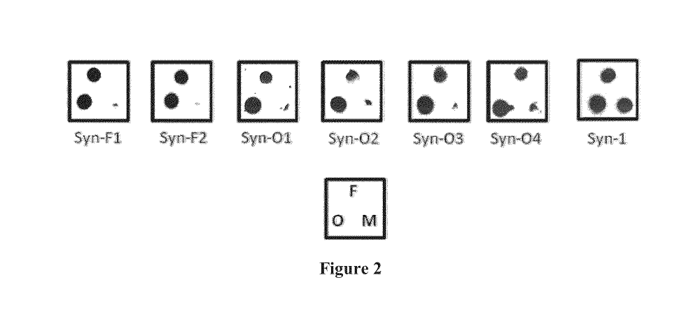

The rest of the dot blots are shown in FIG. 2. Out of 278 clones tested by dot blot, 45 clones were found to be stable and out of that 14 clones were specific for α-synuclein aggregates.

### Example 3

### Mass Culture and Purification of Monoclonal Antibody

Six clones identified to be specific for α-synuclein aggregates were taken for mass culture. These clones were Syn-F1, Syn-F2, Syn-O1, Syn-O2, Syn-O3 and Syn-O4. The growth media was CDM4mAb (Hyclone) supplemented with 2 mM glutamax (Gibco), Penstrep (Sigma), 50 μg/ml Gentamycin(Sigma) and 50 μM beta-ME (Sigma). Once the cells were confluent and the media colour changed to yellow, the culture supernatnants were collected and stored at −20° C. until used. Monoclonal antibodies were purified from the culture supernatants using Protein G-Agarose affinity chromatography. Protein G-Agarose column was prepared and equilibrated with 20 bed volumes of 20 mM Phosphate buffer pH 7.2. The culture supernatant (200 ml) was centrifuged at 1500 rpm for 10 min at 4° C. The supernatants were collected and passed through the column, 5-6 times, keeping the flow rate to ˜1 ml/minute. The column was then washed with 15 bed volumes of 20 mM Phosphate buffer pH 7.2 to remove the proteins. The bound antibody was eluted as 500 μl fractions using elution buffer (50 mM Glycine pH 2.5) into 1.5 ml centrifuge tubes containing 50 μl of neutralization buffer (1M Tris pH 8.0). The purified antibody was finally dialyzed against PBS. Monoclonal antibody purified from different batch was pooled together and the concentration was determined by BCA assay. These monoclonal antibodies were lyophilized and stored as 100 μg aliquots at −20° C. until used.

### Example 4

### Characterization of Purified Antibody

### Inhibition ELISA

A 384 well black maxisorp plate (NUNC) was coated with 50 μl per well (1.4 μg/ml) of α-synuclein fibrils in PBS pH 7.4 by overnight incubation at 4° C. The plate was washed 4× with PBST (PBS with 0.05% Tween) and blocked with blocking buffer (2.25% gelatin in PBS with 0.05% Tween) for 1 h at room temperature. In 0.6 ml siliconized tubes serial double dilutions of the α-synuclein fibrils, oligomers or monomers were made in blocking buffer (starting from 80 μl M followed by 18 serial double dilution). To each tube equal volume of 20ng/ml of the purified monoclonal antibodies (Syn-F1, Syn-F2, Syn-O1, Syn-O2, Syn-O3, Syn-O4) or Syn-1 (BD Bioscience), used as a control, were added, to give a final concentration of 10 ng/ml, and incubated the tubes by mixing for 2.5 h at room temperature. The preincubated solution was then added to the ELISA plates and incubated for 10 minutes at room temperature. After washing the plates with PBST (4×), 1/15000 diluted goat anti-mouse IgG-HRP conjugate (Dako) was added and the plates were incubated for 1 h at room temperature. The plates were washed with PBST and 50 μl of substrate (SuperSignal ELISA Femto Maximum Sensitivity Substrate, Thermo Scientific) was added and immediately read the plate using Victor X3 microtiter plate reader. Taking the log dilution of the α-synuclein concentration on x-axis and the CPS values on the y-axis, graphs were plotted.

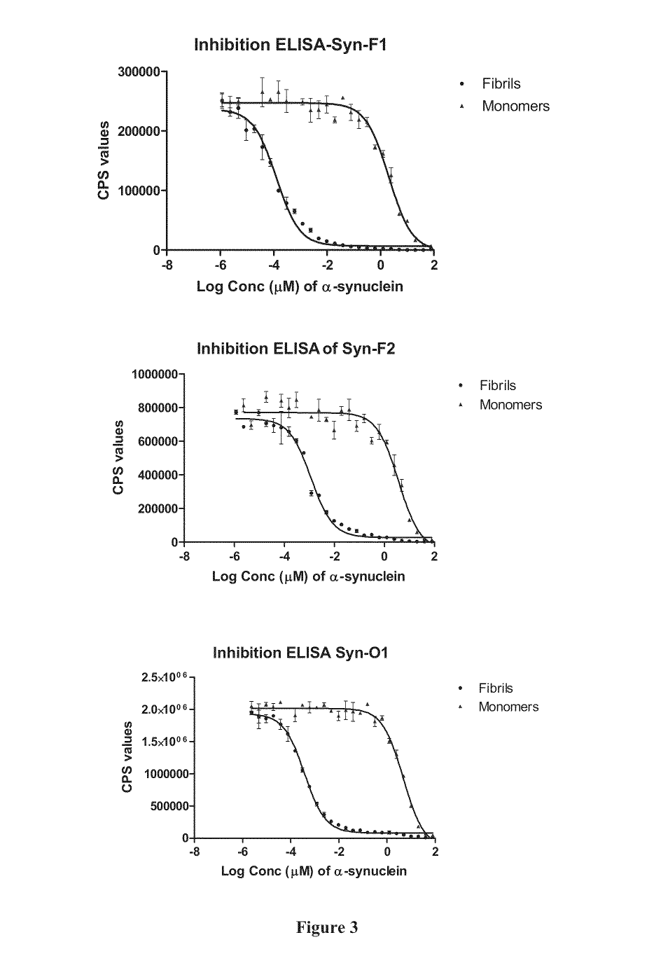

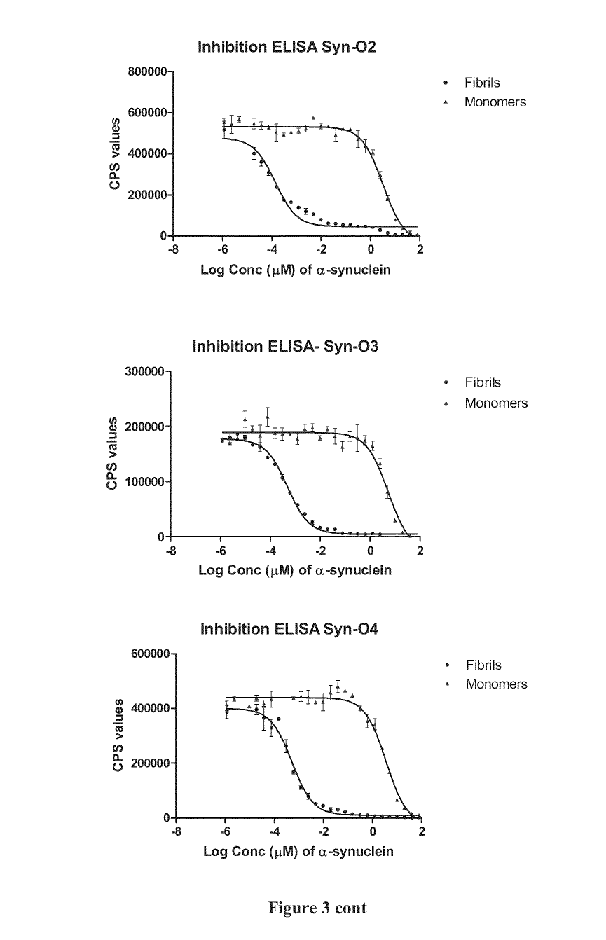

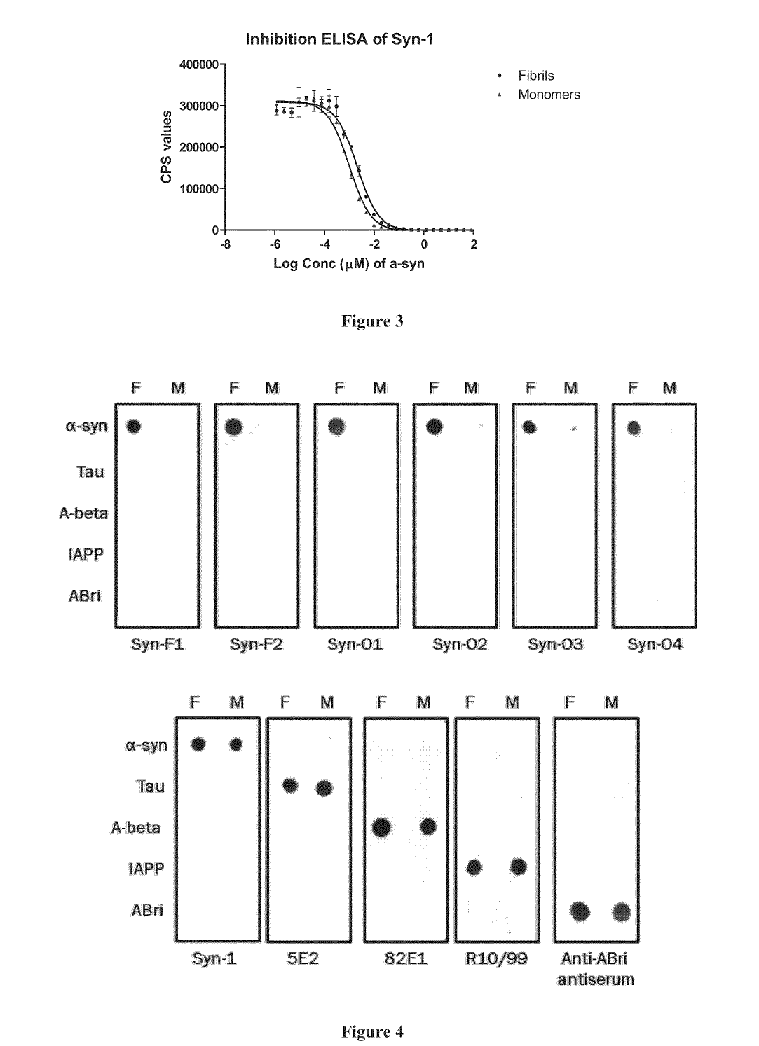

Results for the comparison of binding affinity of mAbs by inhibition ELISA can be found in FIG. 3 and Table 3.

TABLE 3

| | IC 50 (μM) | IC 50 (μM) | Fold Affinity to |
|---|---|---|---|
| mAbs | Fibrils | Monomers | Fibrils |
| Syn-F1 | 0.00012 | 2.083 | 16558 |
| Syn-F2 | 0.00118 | 3.707 | 3141 |
| Syn-O1 | 0.00037 | 4.793 | 12737 |
| Syn-O2 | 0.00012 | 3.283 | 27312 |
| Syn-O3 | 0.00047 | 5.329 | 11129 |
| Syn-O4 | 0.00052 | 3.605 | 6932 |
| Syn-1 | 0.00210 | 0.001 | 0.476 |

All the monoclonal antibodies, Syn-F1, Syn-F2, Syn-O1, Syn-O2, Syn-O3 and Syn-O4, were found to be specific for α-synuclein fibrils.

### Examples 5

### Testing the Cross Reactivity of the Antibodies—Dot Blot

A) To test the cross-reactivity with amyloid fibrils formed by different proteins 50 ng (in 5 μl PBS, pH 7.4) of various amyloid proteins and peptides, fibrils (F) and monomers (M) from α-synuclein, Tau, A-beta, IAPP and ABri were spotted onto a nitrocellulose membrane and dried at room temperature for 30 min. The membranes were blocked with 5% skimmed milk in PBST (PBS containing 0.05% Tween) and incubated for 1 h at room temperature. After washing the membranes 3× with PBST, the membranes were incubated (50 ng/ml in PBST) with monoclonal antibodies, Syn-F1, Syn-F2, Syn-O1, Syn-O2, Syn-O3, Syn-O4, or control antibodies, Syn-1 (BD Bioscience) for α-synuclein, 5E2(Gift from Dr. D. Walsh, Harvard Medical School) for Tau, 82E1 (IBL) for A-beta, R10/99 (Santa Cruz Biotechnology) for IAPP and Rabbit anti-ABri antiserum for Abri, for 2 h at room temperature. The membranes were washed and incubated with either 1/20000 diluted goat anti-mouse IgG-HRP (Jackson ImmunoResearch) or 1/10000 diluted goat anti-rabbit IgG-HRP (Jackson Immunoresearch) for 1 h at room temperature. The blots were then developed with Super signal West Pico chemiluminescent substrate.

The results of the cross-reactivity with amyloid fibrils formed by different proteins can be seen in FIG. 4. The monoclonal antibodies according to the invention were found to be specific for α-synuclein amyloid fibrils and did not cross-react with other amyloid fibrils or monomers.

B) To test the cross reactivity of the conformational monoclonal antibodies, of the invention to amyloid fibrils formed by β- and γ-synuclein. 50 ng of the fibrils (F) and monomers (M) from α-, β- and γ-synuclein were spotted onto a nitrocellulose membrane. The membranes were blocked with 5% skimmed milk in PBST for 1 h and and then probed with our monoclonal antibodies (Syn-F1, Syn-F2, Syn-O1, Syn-O2, Syn-O3, Syn-O4) or control antibodies, Syn-1 (BD Bioscience) for α-synucelin, Anti-Beta-synuclein (Santa Cruz Biotechnology) for beta-synuclein and C-20 (Santa Cruz Biotechnology) for gamma-synuclein for 2 h at room temperature. The membranes were washed and incubated with either 1/20000 diluted goat anti-mouse IgG-HRP conjugate (Jackson Immunoresearch) or 1/300000 diluted chicken anti-goat IgG-HRP conjugate (Santa Cruz Biotechnology) for 1 hour at room temperature. The blots were developed with Super signal West Pico chemiluminescent substrate.

The results of the cross-reactivity with amyloid fibrils formed by β-synuclein or γ-synuclein can be seen in FIG. 5. The monoclonal antibodies according to the invention were found to be specific to α-synuclein fibrils and did not react to either β- or γ-synuclein in the fibril or monomeric form.

C) To test the cross reactivity of our conformational monoclonal antibodies to different fragments of α-synuclein. 50 ng of the fibrils (F) and monomers (M) from the full-length α-synuclein (1-140) and of the peptide fragments, α-synuclein (1-122), NAC (61-95) and NAC (61-78) were spotted onto a nitrocellulose membrane. The membranes were blocked with 5% skimmed milk in PBST for 1 h and probed with our monoclonal antibodies (Syn-F1, Syn-F2, Syn-O1, Syn-O2, Syn-O3, Syn-O4) and control antibodies, Syn-1 (BD Bioscience) for α-synuclein, and 5C2 (Santa Cruz Biotechnology) for 2 h at room temperature. The membranes were washed before incubating with either 1/20000 diluted goat anti-mouse IgG-HRP conjugate (Jackson Immunoresearch) or 1/300000 diluted chicken anti-goat IgG-HRP conjugate (Santa Cruz Biotechnology) for 1 h at room temperature. The blots were then developed with Super signal West Pico chemiluminescent substrate.

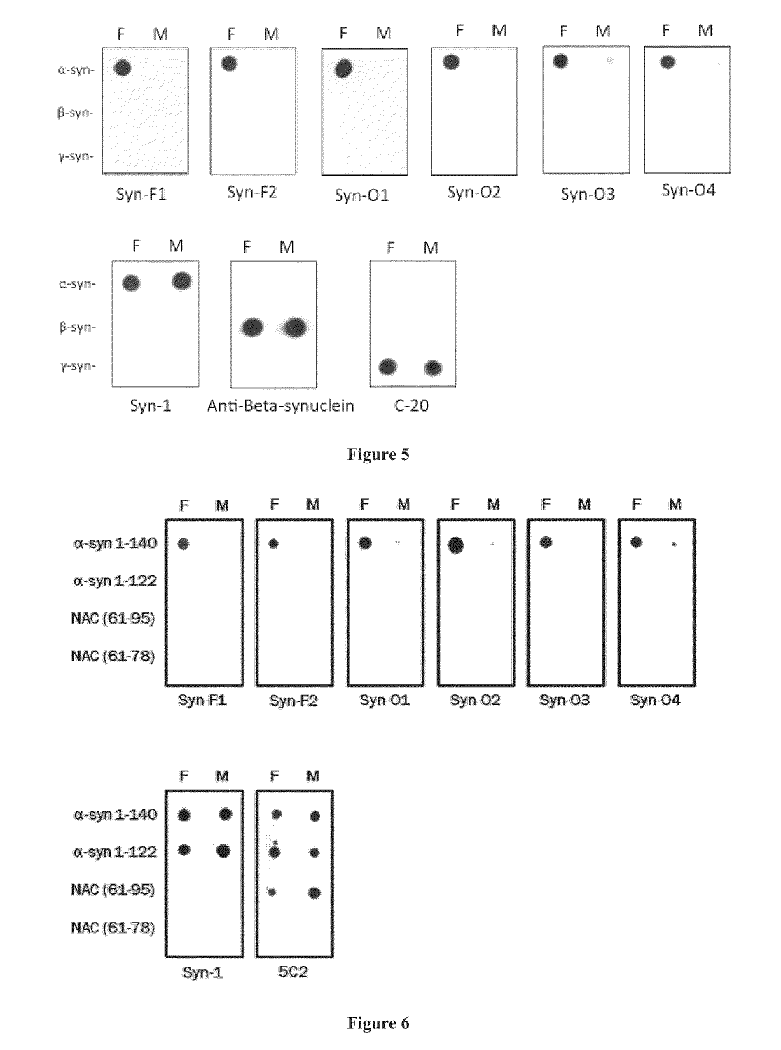

The results of the cross-reactivity with amyloid fibrils formed by different fragments of α-synuclein can be seen in FIG. 6. The monoclonal antibodies according to the invention did not react with amyloid fibrils formed by any of the truncated α-synuclein (1-122), NAC region (61-95) or NAC (61-78) but reacted only to the fibrils formed by the full length α-synuclein (1-140) suggesting that a part of epitope may be in the C-terminal region of α-synuclein. The non-reactivity of our mAbs to α-synuclein monomers also suggests that the epitope recognized by our mAb's may be conformation specific.

### Example 6

### Determination of Kinetic Constants

### Biacore Analysis

The kinetic constants for the interaction between our mAb's and -synuclein fibrils were determined by surface plasmin resonance measurements using the BIAcore X 100 instrument.

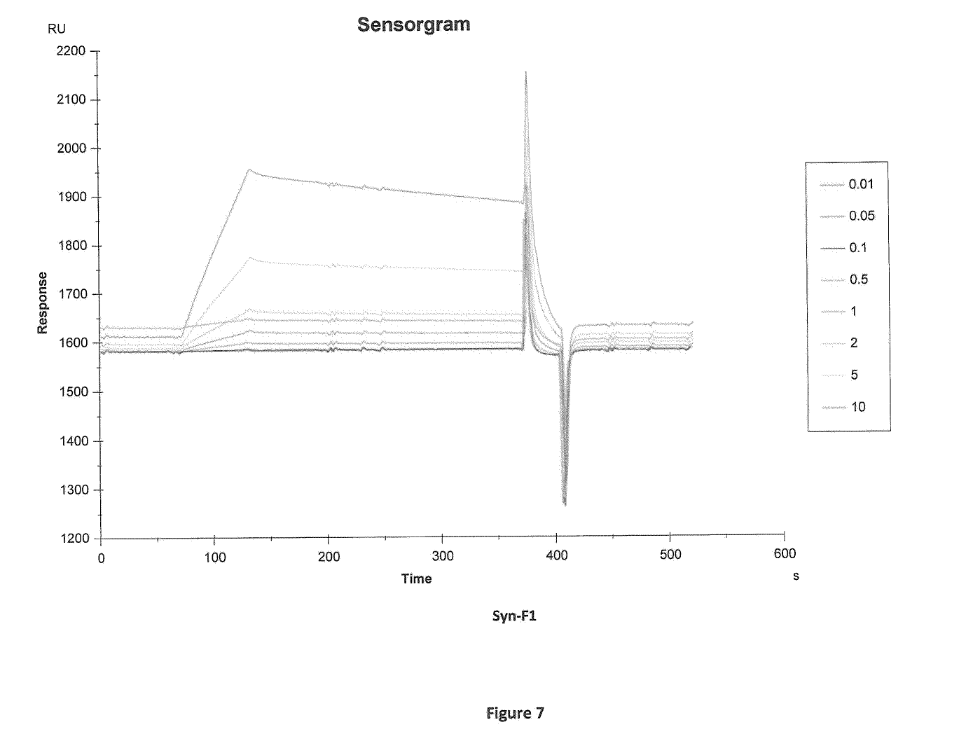

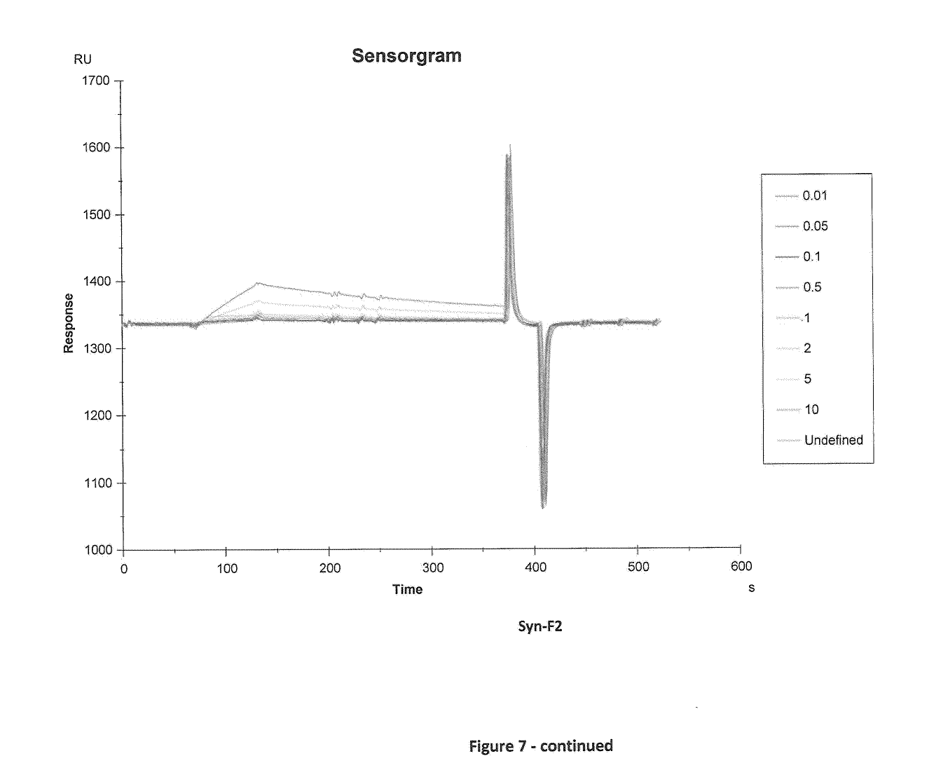

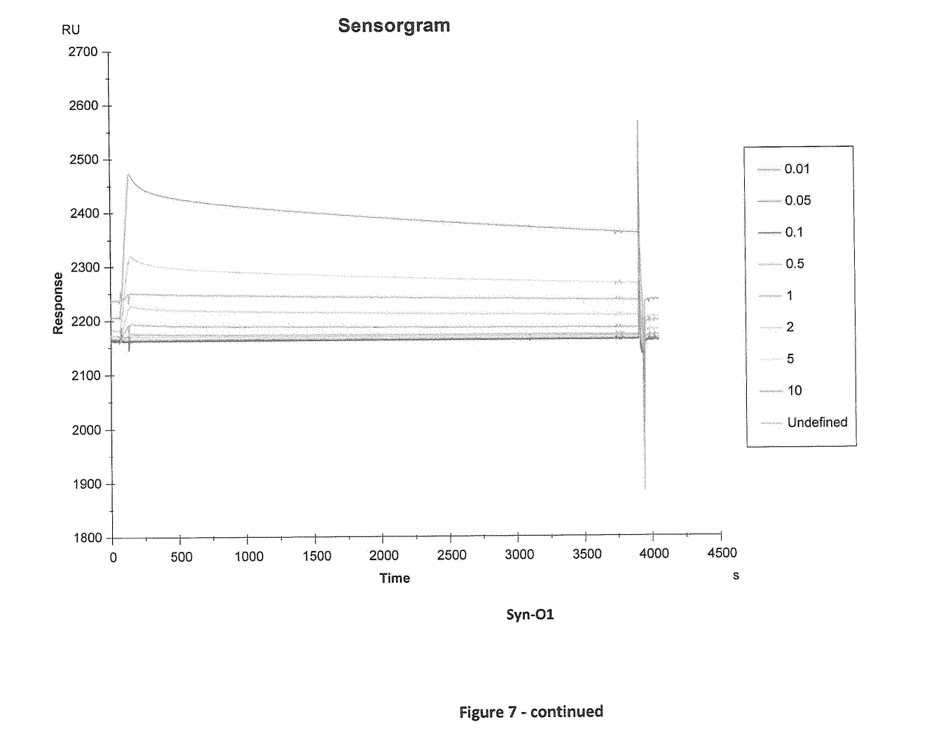

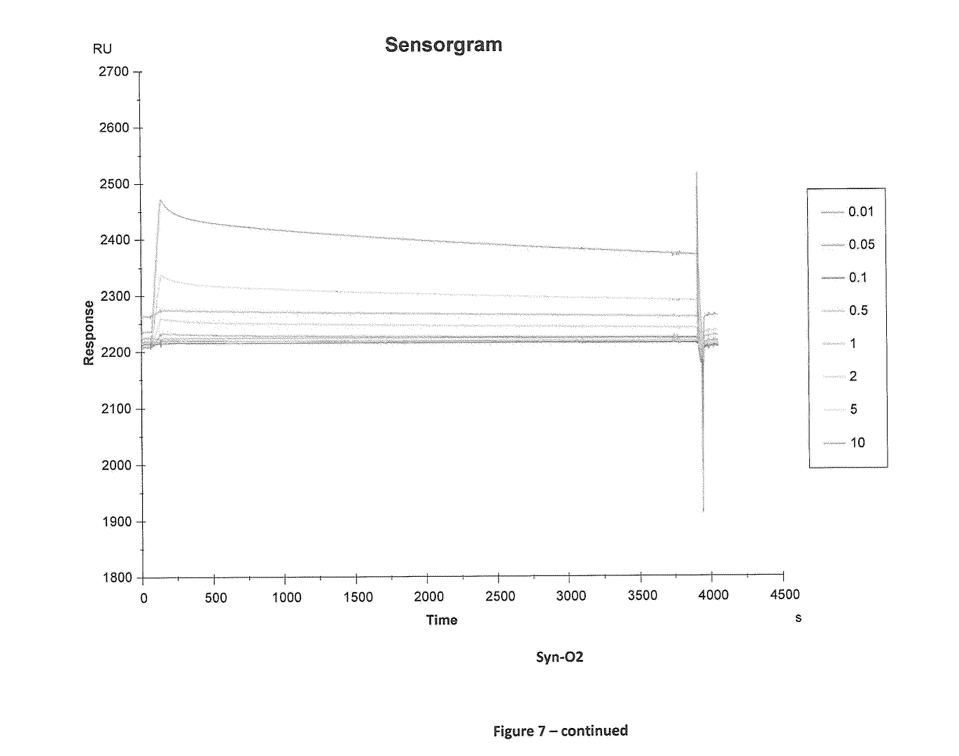

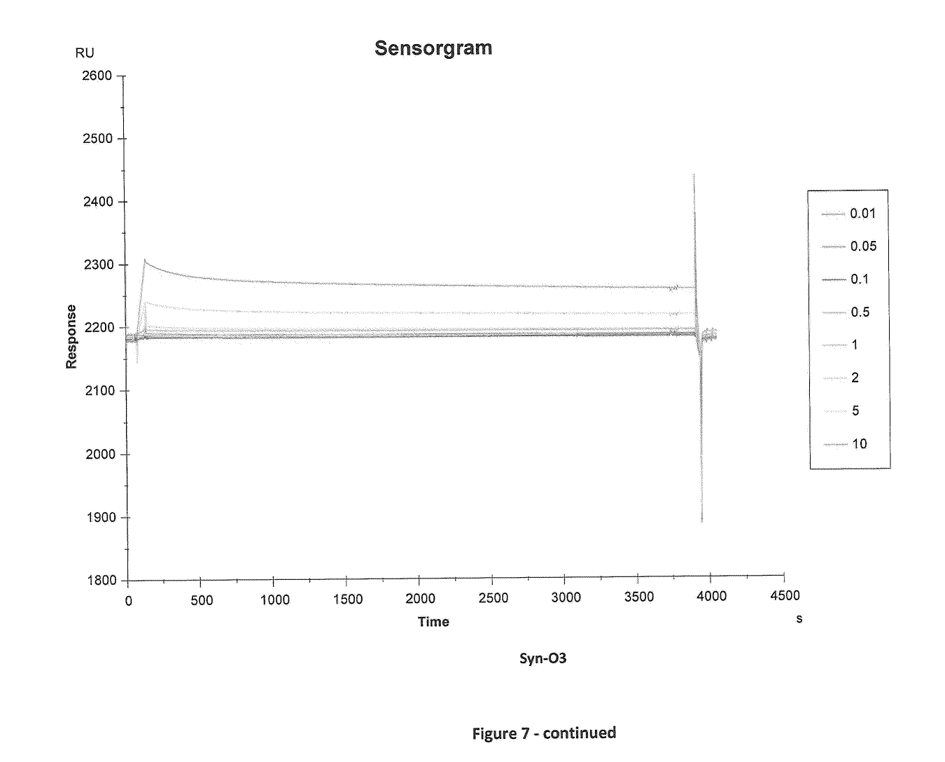

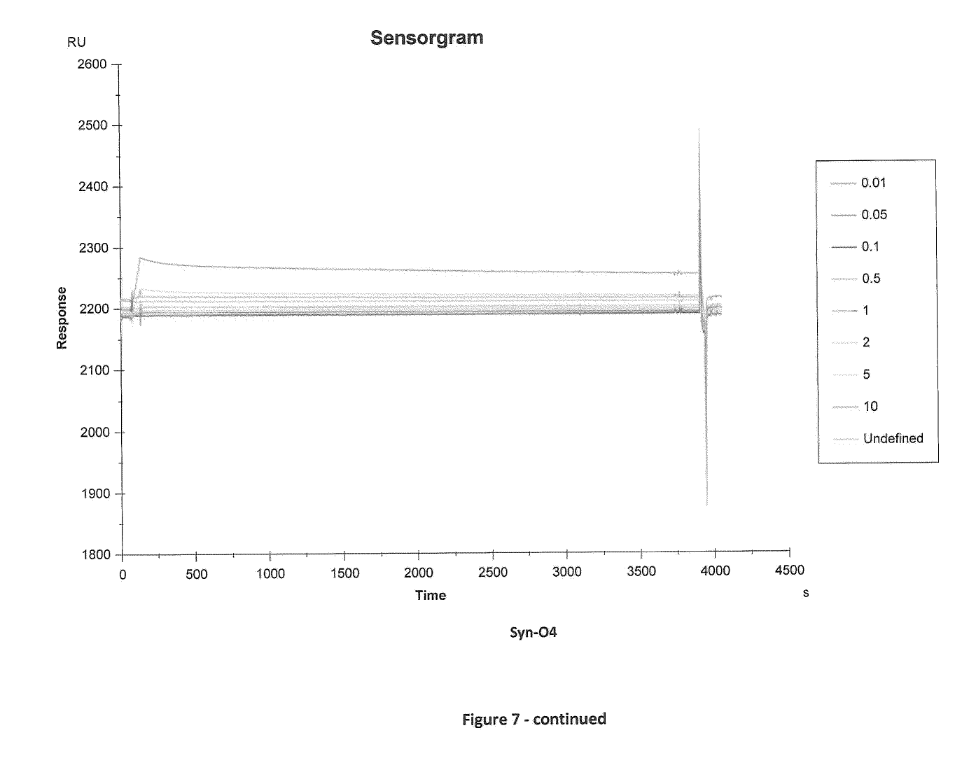

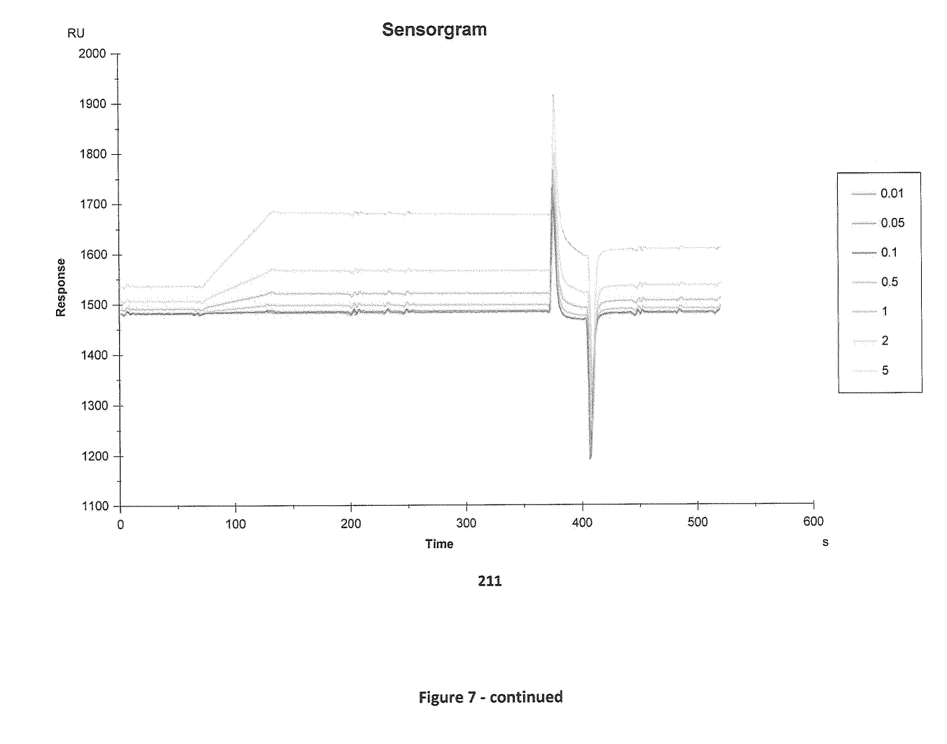

Alpha-synuclein fibrils were immobilized to a CM5 sensor chop activated with N-hydroxy succinimide (NHS) and N ethyl-N (dimethylaminopropyl) carbofiimide (EDC) by injecting 35 μl of 120 μg/ml-synuclein fibrils in 10 mM Sodium acetate buffer, pH 4.5. The association rate constant was obtained by injecting eight different concentrations of the mAbs (Syn-F1, Syn-F2, Syn-O1, Syn-O2, Syn-O3, Syn-O4 and mAb 211 (Santa Cruz Biotechnolgy)) ranging from 0.01 to 10 nM in HBS running buffer (10 mM hepes, 0.15 m NaCl, 3.4 mM EDTA and 0.005% surfactant P20; pH 7.4) at a flow rate of IOW/min. The dissociation rate was measured at flow rate of 40 μl/min. The sensor chip was regenerated using 100 mM NaHCO3 pH. 9.6. Sensograms were analysed with the BOA evaluation software. The results showing the comparison of mAbs by Biacore can be seen in FIG. 7 and Table 4. Monoclonal antibodies have a Kd of less than 10−8 were found. mAb Syn-O1 was found to be having the highest affinity of KD15.9 pM. The lowest affinity among the 6 mAbs were found to be for Syn-F2 with 2.6 nM.

TABLE 4

| mAb | ka (1/Ms) | kd (1/s) | KD (M) |
|---|---|---|---|
| Syn-F1 | 1.230E+7 | 0.001564 | 1.272E−10 |
| Syn-F2 | 1.256E+6 | 0.003377 | 2.688E−9 |
| Syn-O1 | 5.857E+6 | 9.360E−5 | 1.598E−11 |
| Syn-O2 | 1.314E+7 | 0.001274 | 9.694E−11 |
| Syn-O3 | 2.217E+6 | 3.789E−4 | 1.709E−10 |
| Syn-O4 | 7.236E+6 | 9.964E−4 | 1.377E−10 |
| mAb 211 | 1.648E+5 | 4.220E−4 | 2.560E−9 |

### Example 7

### Epitope Mapping for the mAbs

In order to find out if the mAbs of the invention detect linear or conformational epitopes, affinity of the antibodies was tested against a peptide library covering α-synuclein sequence. Peptides of 14 amino acid long, with 7 amino acid overlap, covering the human α-synuclein sequence (see Table 5) were synthesized (Shanghai Hanhong Chemical Co., China). The peptides were dissolved in either autoclaved water or DMSO to give a final concentration of 1 mg/ml.

TABLE 5

| Peptide | Sequence | |
|---|---|---|
| No. | no. | Peptide Sequence |
| 1 | 1-14 | H-MDVFMKGLSKAKEG-OH |
| 2 | 8-21 | H-LSKAKEGVVAAAEK-OH |
| 3 | 15-28 | H-VVAAAEKTKQGVAE-OH |
| 4 | 22-35 | H-TKEQGVAEAAGKTKE-OH |
| 5 | 29-42 | H-AAGKTKEGVLYVGS-OH |
| 6 | 36-49 | H-GVLYVGSKTKEGVV-OH |
| 7 | 43-56 | H-KTKEGVVHGVATVA-OH |
| 8 | 50-63 | H-HGVATVAEKTKEQV-OH |
| 9 | 57-70 | H-EKTKEQVTNVGGAV-OH |
| 10 | 64-77 | H-TNVGGAVVTGVTAV-OH |
| 11 | 71-84 | H-VTGVTAVAQKTVEG-OH |
| 12 | 78-91 | H-AQKTVEGAGSIAAA-OH |
| 13 | 85-98 | H-AGSIAAATGFVKKD-OH |
| 14 | 92-105 | H-TGFVKKDQLGKNEE-OH |
| 15 | 99-112 | H-QLGKNEEGAPQEGI-OH |
| 16 | 106-119 | H-GAPQEGILEDMPVD-OH |
| 17 | 113-126 | H-LEDMPVDPDNEAYE-OH |
| 18 | 120-133 | H-PDNEAYEMPSEEGY-OH |
| 19 | 127-140 | H-MPSEEGYQDYEPEA-OH |

### Pepscan by Dot Blot

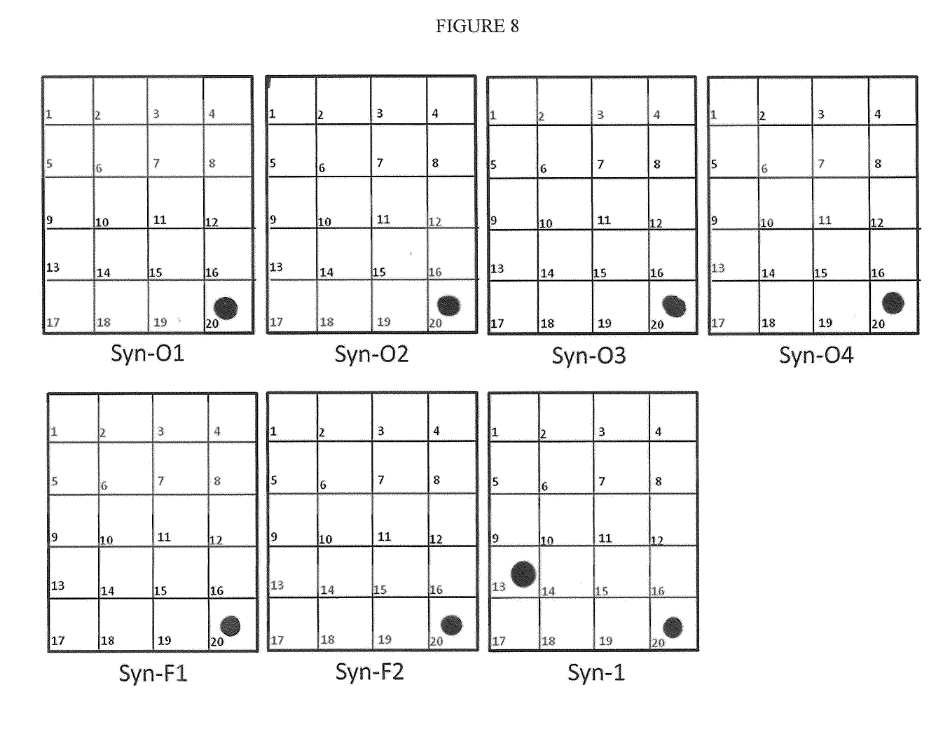

Nitrocellulose membrane was pretreated with 0.1% EDC (1-Ethyl-3-[3-dimethylaminopropyl] carbodiimide hydrochloride), in PBS, for 10 min at room temperature and air-dried. 200 ng (in 5 μl PBS, pH 7.4) of the peptides 1-19 (α-synucelin peptide library, table 5) and 50 ng of the α-synuclein fibrils (spot 20) were spotted onto the membrane.—After air-drying the membranes were blocked with 5% skimmed milk in PBST and incubated for 1 h at room temperature. The membranes were washed three times with PBST before incubating (50 ng/ml in PBST) with Syn-F1, Syn-F2, Syn-O1, Syn-O2, Syn-O3, Syn-O4 or Syn-1 monoclonal antibodies and incubated for 2 h at room temperature. The membranes were washed with PBST and incubated with 1:20000 diluted (in PBST) goat anti-mouse IgG-HRP (Jackson immunoResearch). The blots were developed using Super signal West Pico chemiluminescent substrate (Thermo Scientific). The results of the pepscan be seen in FIG. 8.

The pepscan results using dot blot suggested that the mAbs, Syn-O1, Syn-O2, Syn-O3, Syn-O4, Syn-F1, or Syn-F2, do not recognize a linear epitope.

### Pepscan by ELISA

To confirm the pepscan results using dot blot the mAbs were also tested by Pepscan using ELISA. A 384 well black maxisorp plate (NUNC) was coated with 50 μl (500 ng/well) of peptides (α-synuclein peptides 1-19 as detailed in Table 5) in 0.2 M NaHCO₃ pH 9.6 and 50 μl (100 ng/well) α-synuclein fibrils in PBS pH 7.4 by overnight incubation at 37° C. allowing for complete drying. The plate was washed three times with PBST and blocked with 100 μl of blocking buffer (2.25% gelatin in PBST) for 1 h at room temperature. After washing the plate three times with PBST, 50 μl (100 ng/ml) of the mAbs (Syn-F1, Syn-F2, Syn-O1, Syn-O2, Syn-O3 and Syn-O4), and the control antibodies (50 μl (100 ng/ml) of Syn-1 (Mouse anti-α-synuclein; BD Bioscience), 50 μl (100 ng/ml) of N-19 (Goat anti-α-synuclein; Santa Cruz Biotechnology), 50 μl (100 ng/ml) of 211 (Mouse anti-α-synuclein; Santa Cruz Biotechnology), 50 μl (200 ng/ml) of FL-140 (Rabbit anti-α-synuclein; Santa Cruz Biotechnology), 50 μl (1 μg/ml) of 5C2 (Mouse anti-α-synuclein; Santa Cruz Biotechnology) and 50 μl (1 μg/ml) of 3B6 (Mouse anti-α/β-synuclein; Santa Cruz Biotechnology) were added and incubated for 1 h at room temperature. The plate was washed with PBST and either goat anti-mouse IgG-HRP conjugate (1/20000; Jackson ImmunoResearch) or goat anti-rabbit IgG-HRP (1/5000; Jackson Immunoresearch) conjugate or diluted chicken anti-goat IgG-HRP conjugate (1/20000) were added and incubated the plates for 1 h at room temperature. The plate was washed with PBST and 50 μl of substrate (SuperSignal ELISA Femto Maximum Sensitivity Substrate, Thermo Scientific) was added and immediately read the plate using Victor X3 microtiter plate reader. After the ELISA signals were measured, the plate was also exposed onto X-ray film to develop the spots.

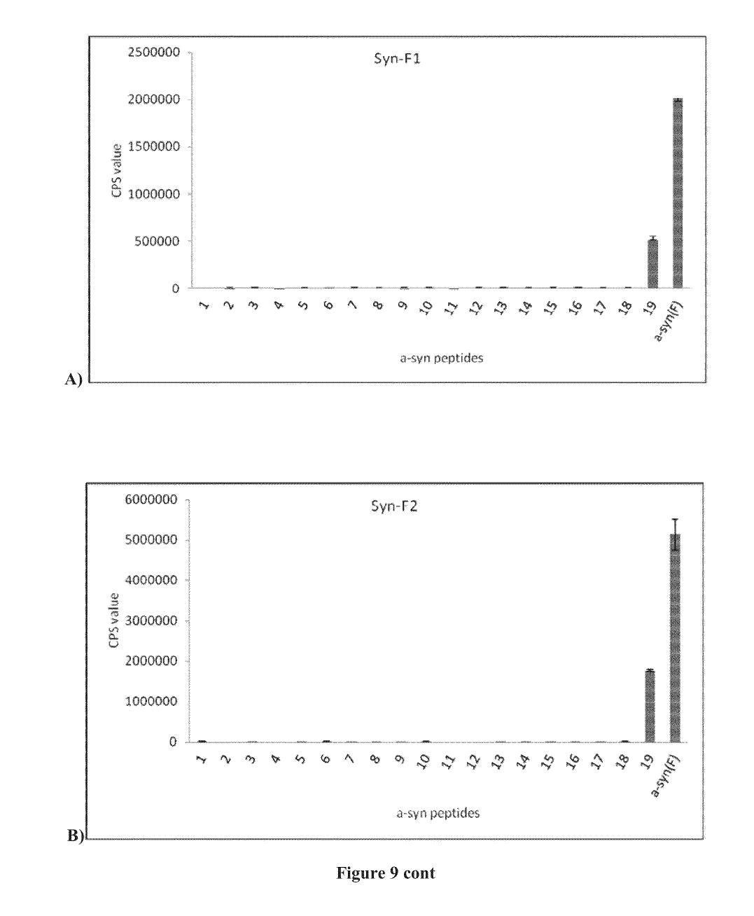

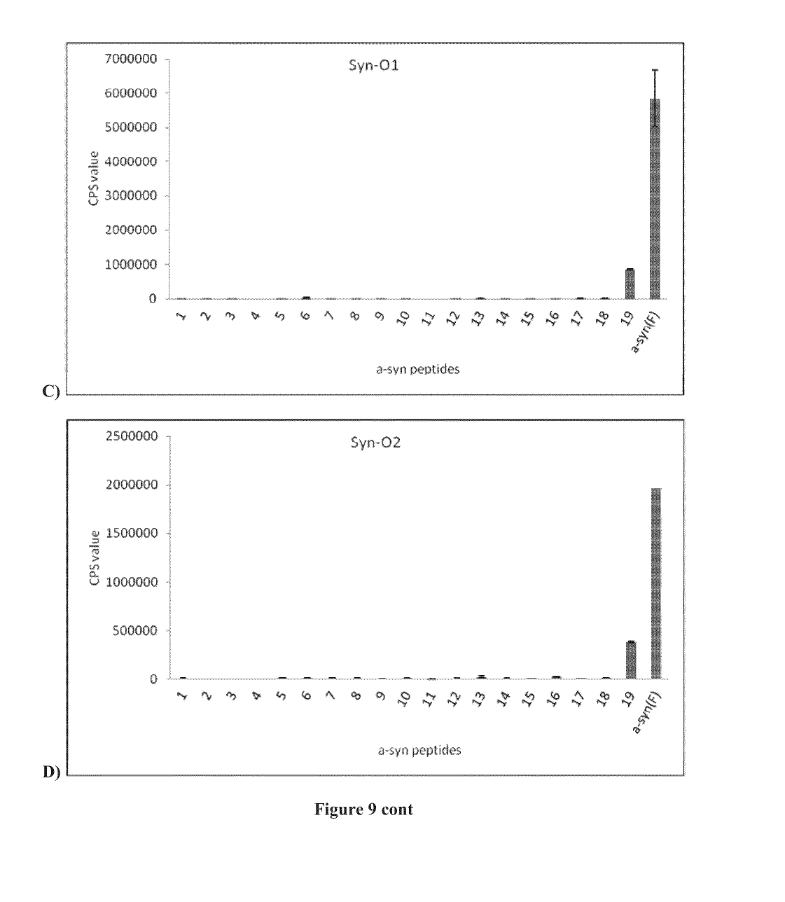

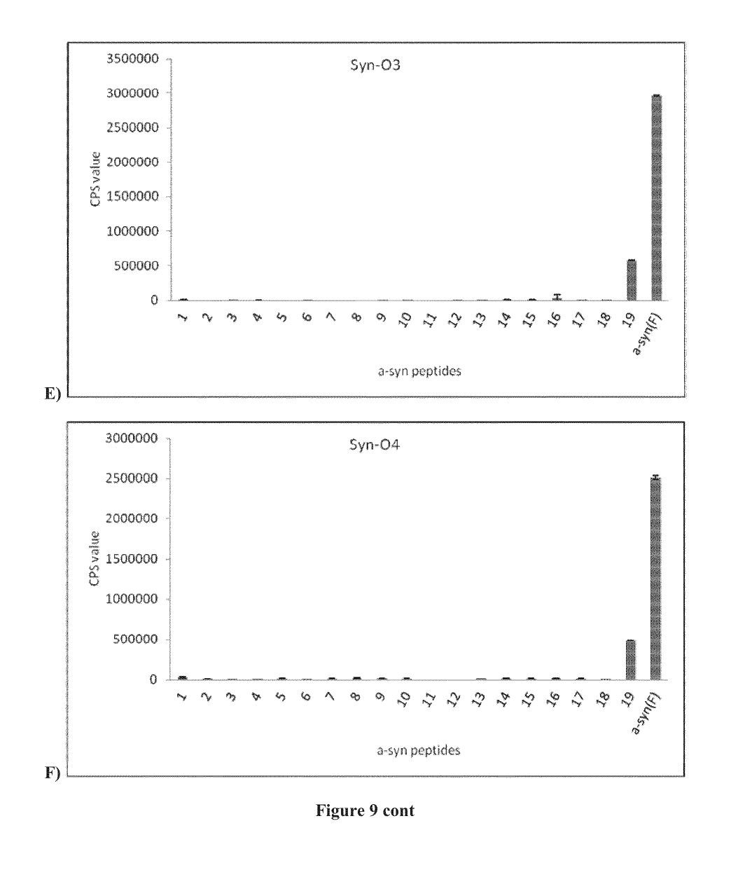

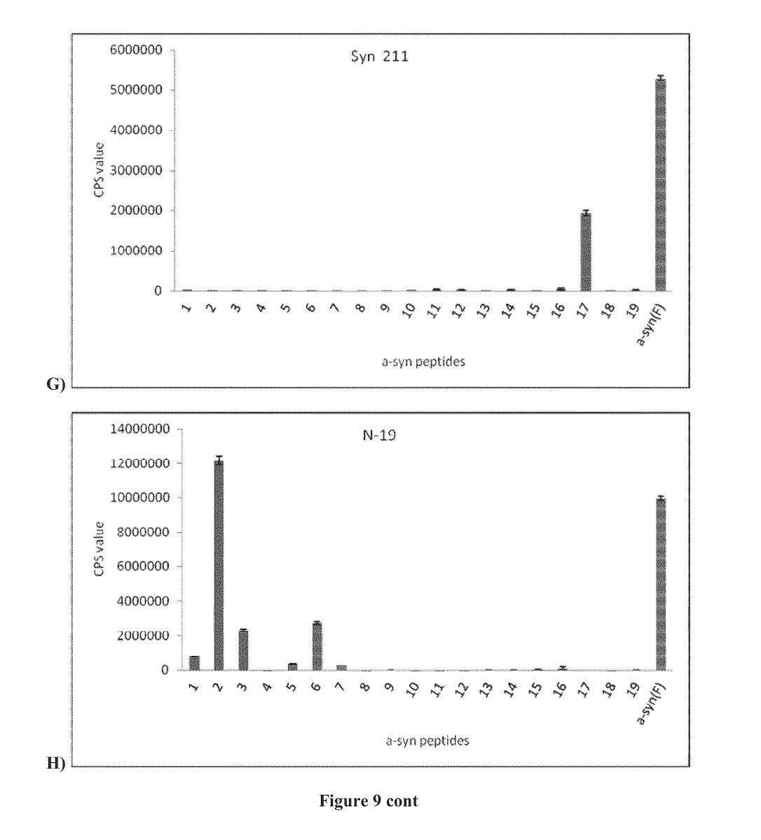

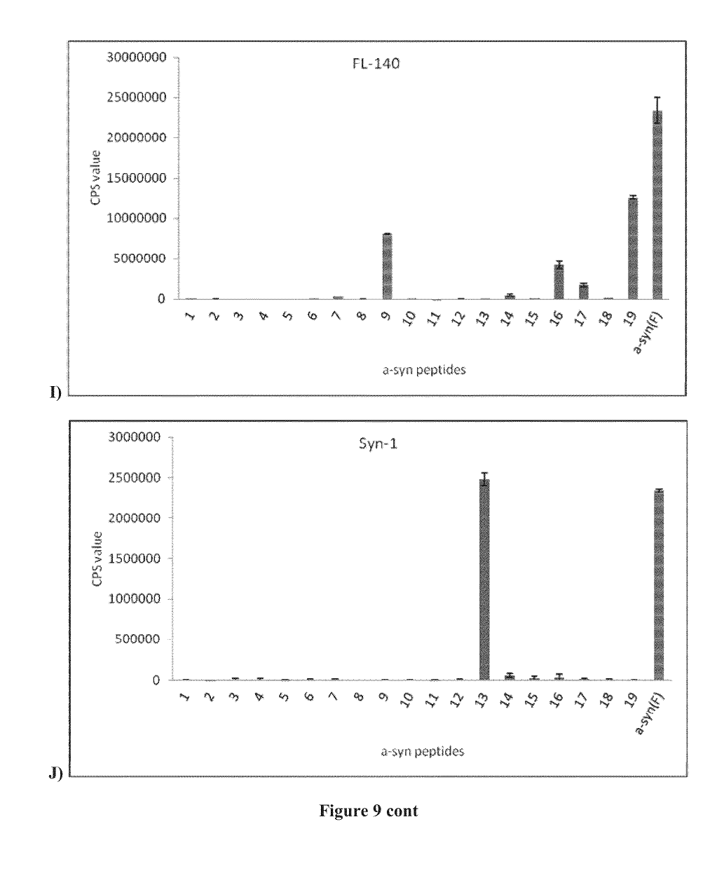

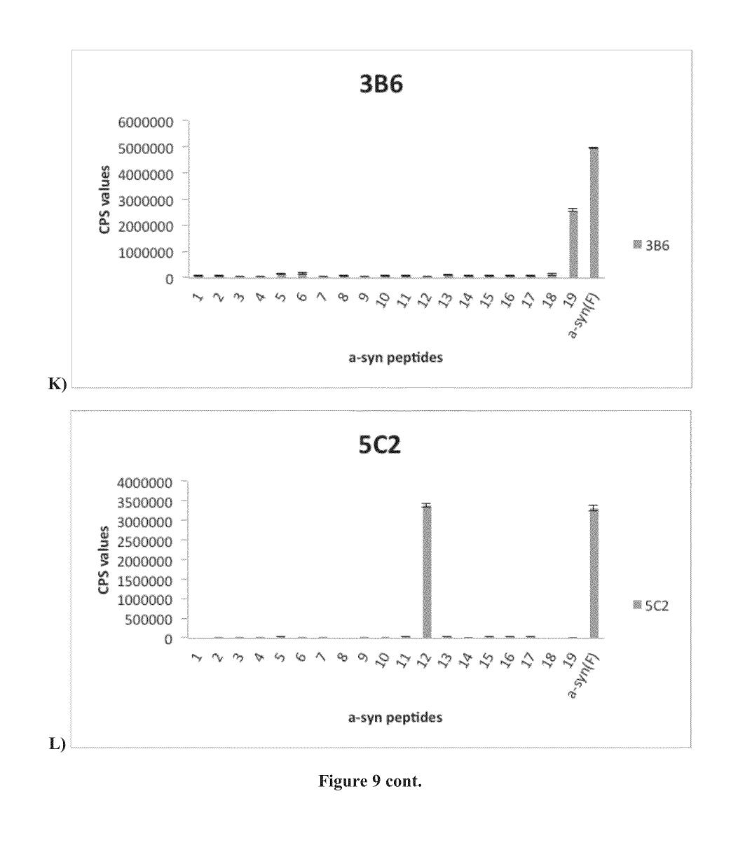

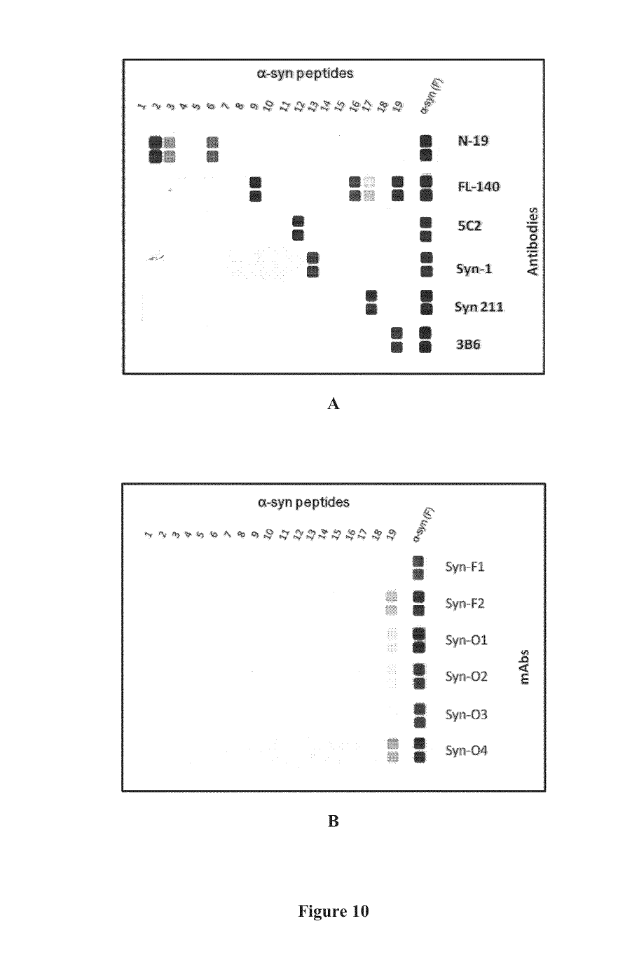

The results of the Pepscan screening by ELISA can be seen in FIGS. 9 and 10 (control antibodies, A, antibodies of the invention, B). The pepscan method by ELISA also showed that the antibodies did not react to any of the peptides but reacted slightly with the C-terminal peptide (127-140) suggesting an involvement of the C-terminal region in forming the conformational epitope.

### Example 8

### Sandwich ELISA to Detect Alpha-Synuclein Aggregates

The 384 well ELISA black microplate (Nunc Maxisorb, NUNC, Denmark) was coated by overnight incubation at 4° C. with 0.1 μg/ml of one of our conformational monoclonal antibodies (Syn-F1, Syn-F2, Syn-O1, Syn-O2, Syn-O3 or Syn-O4) (50 μl/well), in 200 mM NaHCO₃, pH 9.6.

The plate was washed four times with PBS containing 0.05% Tween-20 (PBST), and incubated with 100 μl/well of blocking buffer (PBST containing 2.5% gelatin and 1% BSA) for 2 h at 37° C.

The plate was washed four times with PBST and 50 μl of various concentrations (10, 5, 1, 0.5, 0.1, and 0.05 nM in PBS) of monomeric, oligomeric or aggregated α-synuclein were added. The plate was then incubated at 37° C. for 2.5 h.

After washing four times with PBST, 50 μl of FL-140 (rabbit polyclonal antibody, Santa Cruz Biotechnology) diluted to 1:1000 in blocking buffer was added, and incubated at 37° C. for 2 h.

After the wells were washed 4 times with PBST and 50 μl/well of Goat anti Rabbit IgG HRP (Jackson ImmunoResearch) diluted 1:10,000 in blocking buffer were added and incubated for 1.5 h at 37° C.

The plate was washed 4 times with PBST, and bound HRP activity was assayed by chemiluminescent reaction using an enhanced chemiluminescent substrate (SuperSignal ELISA Femto, Pierce Biotechnology), after which chemiluminescence in relative light units was immediately measured with a PerkinElmer microplate reader.

To confirm that the conformational mAbs detected only the aggregated forms of α-synuclein and not the monomeric forms of α-synuclein, a further ELISA assay as described above was carried out as a control using Syn-1 as the capture antibody.

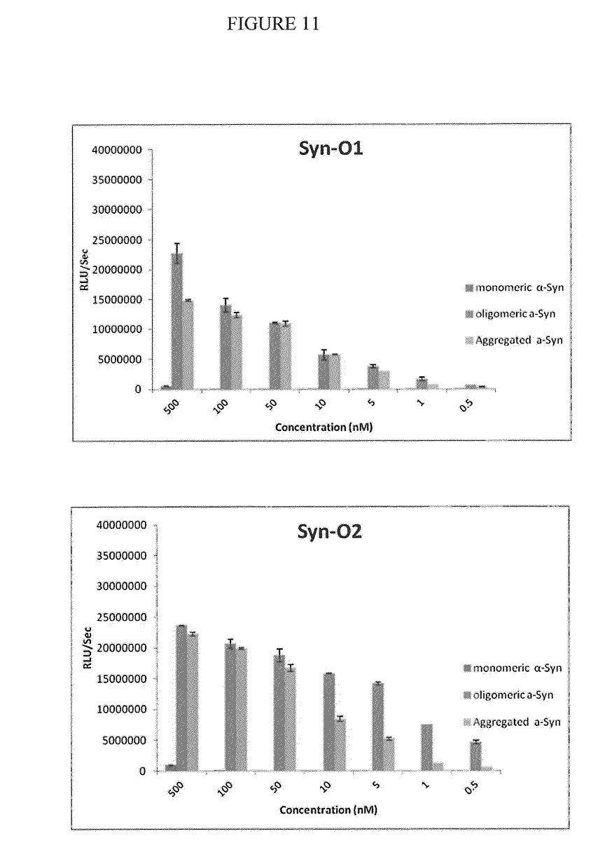

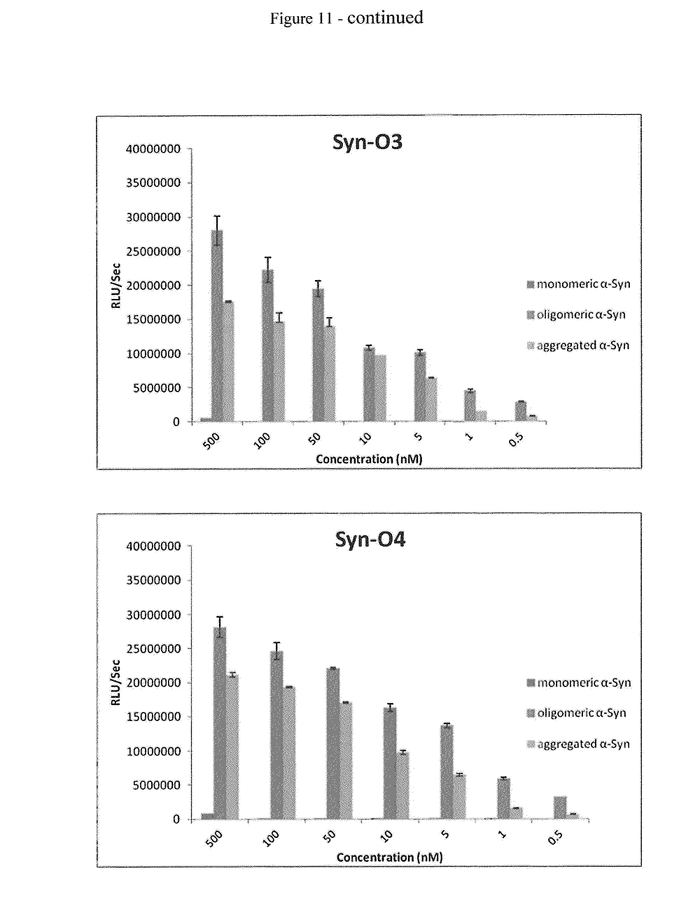

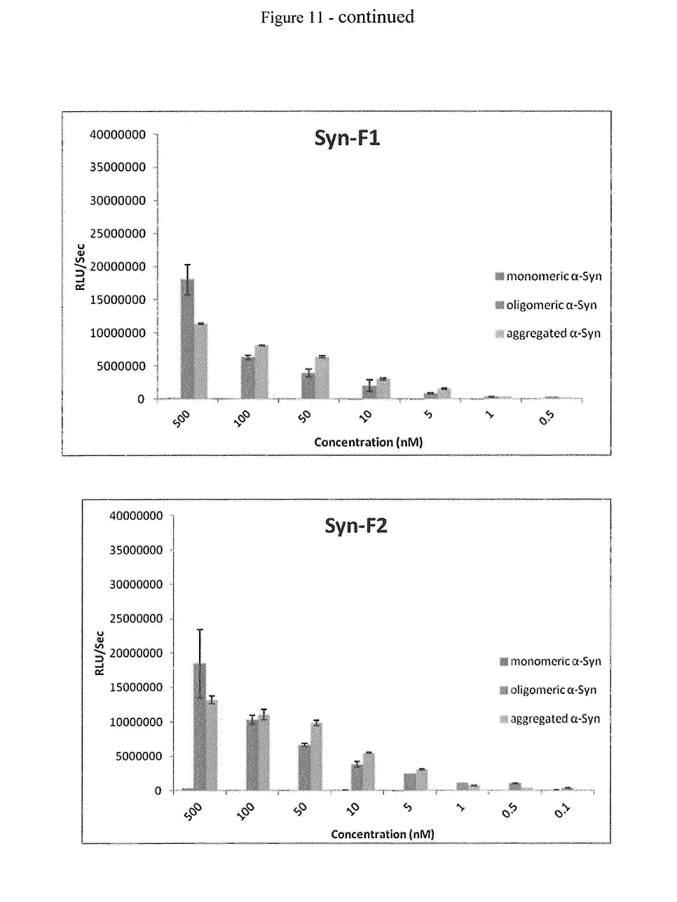

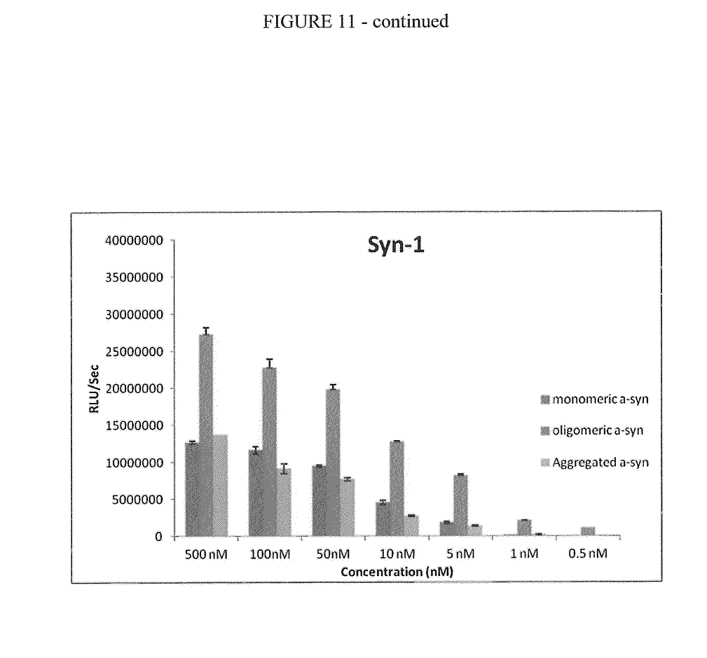

The results are shown in FIG. 11. The results shows that the confirmation mAbs Syn-F1, Syn-F2, Syn-O1, Syn-O2, Syn-O3, and Syn-O4. React to both oligomeric and aggregated forms of α-synuclein. Syn-1 reacts to both monomeric and the aggregated forms of α-synuclein.

Whilst the ELISA shows no detection of the monomeric form of α-synuclein and detection of both the oligomeric and fibril forms of α-synuclein by all the mAbs, the results of the ELISA showed that for Syn-F1 and Syn-F2 there was some specificity towards α-synuclein fibrils over oligomeric forms of α-synucleins.

### Example 9

### Sandwich ELISA to Detect Aggregated Phosphorylated Ser129-Alpha-Synuclein (p-S129-α-Syn

The 384 well ELISA black microplate (Nunc Maxisorb, NUNC, Denmark) was coated by overnight incubation at 4° C. with 0.1 μg/ml of one of our conformational monoclonal antibodies (Syn-F2, Syn-O2) (50 μl/well), in 200 mM NaHCO₃, pH 9.6.

The plate was washed four times with PBS containing 0.05% Tween-20 (PBST), and incubated with 100 μl/well of blocking buffer (PBST containing 2.5% gelatin and 1% BSA) for 2 h at 37° C.

The plate was washed four times with PBST and 50 μl of various concentrations (1, 0.5, 0.1, 0.05, 0.01, 0.005 nM in PBS) of monomeric p-S129-α-syn or aggregated p-S129-α-syn were added. The plate was then incubated at 37° C. for 2.5 h.

After washing four times with PBST, 50 μl of rabbit anti-p-S129-α-synuclein (Epitomics) diluted to 1:1000 in blocking buffer was added, and incubated at 37° C. for 2 h.

After the wells were washed 4 times with PBST and 50 μl/well of Goat anti Rabbit IgG HRP (Jackson ImmunoResearch) diluted 1:10,000 in blocking buffer were added and incubated for 1.5 h at 37° C.

The plate was washed 4 times with PBST, and bound HRP activity was assayed by chemiluminescent reaction using an enhanced chemiluminescent substrate (SuperSignal ELISA Femto, Pierce Biotechnology), after which chemiluminescence in relative light units was immediately measured with a PerkinElmer microplate reader.

To confirm that the conformational mAbs detected only the aggregated forms of α-synuclein and not the monomeric form of α-synuclein, a further ELISA assay as described above was carried out as a control using Syn-1 as the capture antibody.

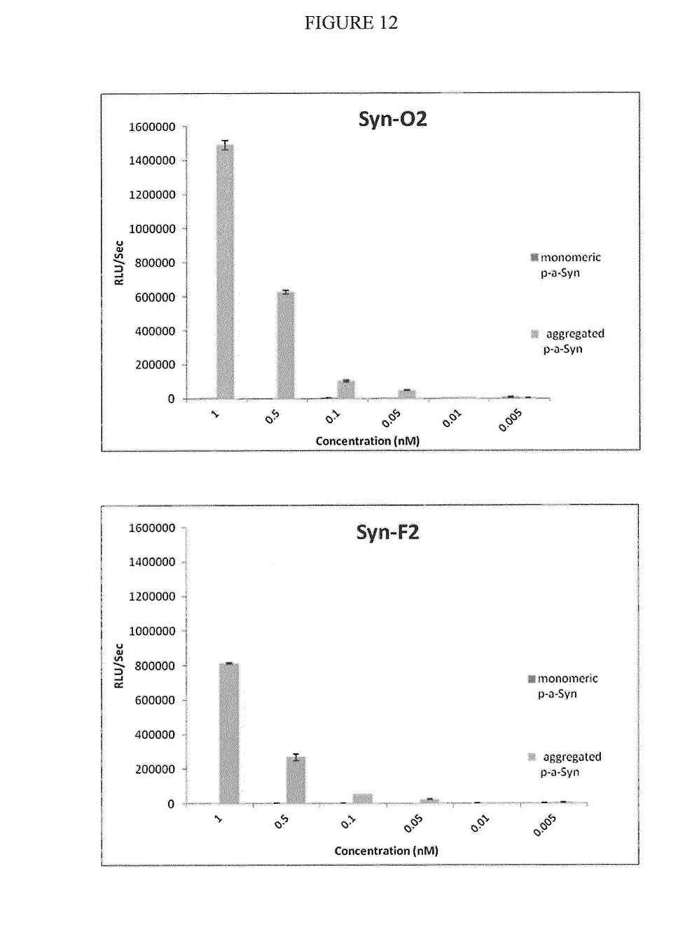

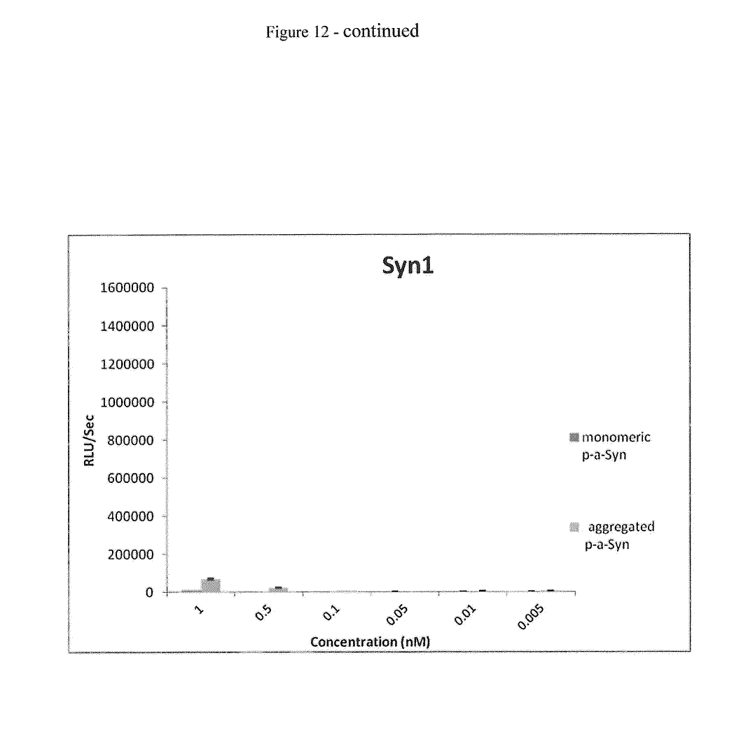

The results can be seen in FIG. 12. The results shows that the confirmation mAbs Syn-F2 and Syn-O2, react to aggregated phosphorylated α-synuclein forms. Syn-1 showed a similar but in both cases low reaction to both monomeric and the aggregated forms of phoshorylated S129 α-synuclein.

The aggregation and phosphorylation of synuclein is thought to play a critical role in PD pathogenesis. Therefore detection of phosphorylated or non-phosphorylated forms of α-synuclein in biological fluids can be used as a potential biological marked for PD.

An ELISA assay has been developed that can specifically detect the aggregated forms of α-synuclein and does not detect the monomeric forms of α-synuclein. The assay uses the antibodies Syn-F1, Syn-F2, Syn-O1, Syn-O2, Syn-O3, or Syn-O4 as the capture antibodies, followed by detection with antibodies such as FL-140 (a rabbit polyclonal antibody raised against full length α-synuclein, Santa Cruz Biotechnology) for aggregated α-synuclein, or a rabbit anti-p-S129-α-synuclein (Epitomics) for aggregates of p-S129-α-synuclein.

### Example 10

### Immunohistochemistry

The mAbs were tested using peroxidase immunohistochemistry in brain sections from Parkinson's disease case. Syn-F1 and Syn-O2 were used at 1 in 10000 (0.1 μg/ml) dilution thereas Syn-F2, Syn-O1, Syn-03 and Syn-O4 were used at 1 in 5000 (0.2 μg/ml) dilution. A mouse negative control slide was also processed simultaneously, where all immunohistochemical steps were included but the primary antibody was omitted.

### Results

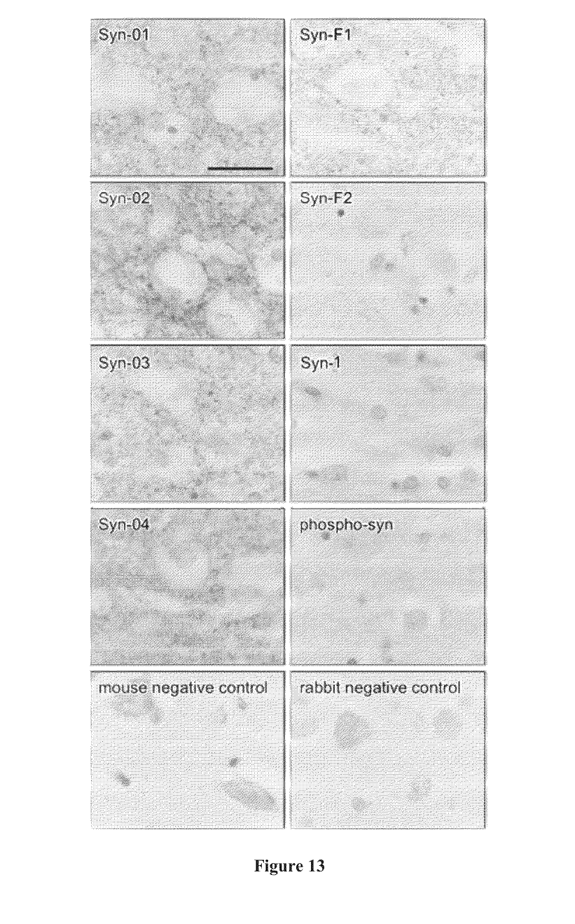

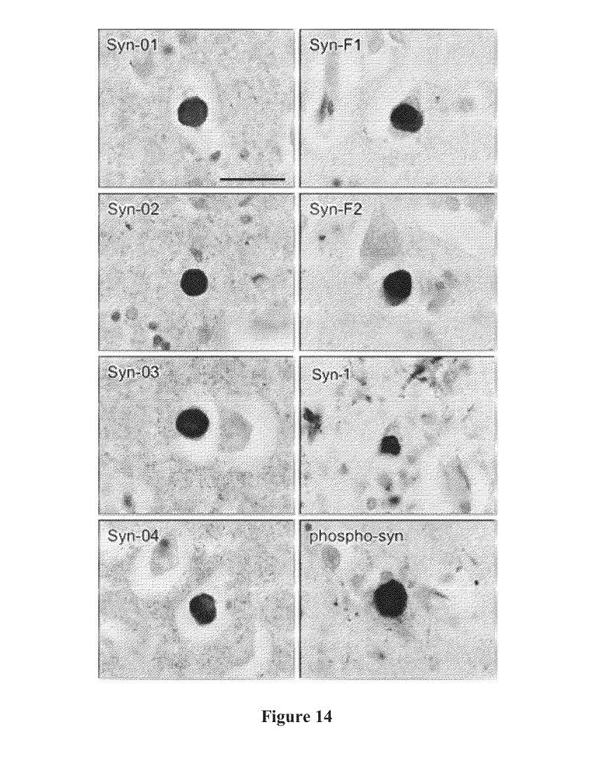

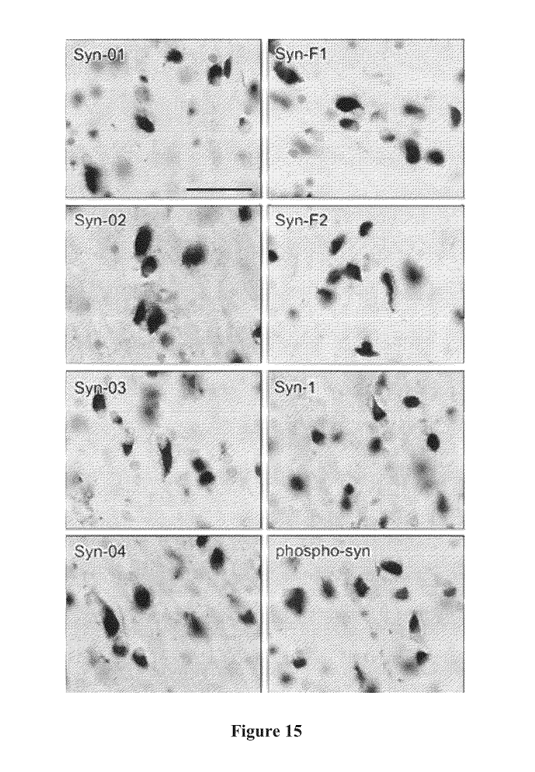

The mAbs have been worked up using peroxidase immunohistochemistry in a Parkinson's disease (PD) and Multi-system atrophy (MSA) cases. All antibodies produced intense immunostaining in Lewy bodies and Lewy neuritis in PD brains (FIG. 14), and glial cytoplasmic inclusions in white matter oligodendrocytes of multiple system atrophy brains (FIG. 15). A mouse negative control slide was also processed and did not produce any immunoreactivity in the section (FIG. 13).

In the control cases (FIG. 13) the images show sections immunostained for our mAbs and the commercially available mouse monoclonal anti-α-synuclein antibody (Syn-1) or mouse monoclonal anti-phosphorylated Ser129 α-synuclein (phospho-syn) in the anterior cingulate cortex from a control case. A mouse and rabbit negative control slide were also processed simultaneously. All sections were counterstained with Cresyl violet (blue) to visualise cell nuclei and membranes. The same adjustments to brightness were applied to all images. Scale bar represents 20 μm (applies to all panels).

In the Parkinson's disease case (FIG. 14) images shows Lewy bodies immunoreactive for our mAbs and the commercially available mouse monoclonal anti-α-synuclein antibody (Syn-1) or mouse monoclonal anti-phosphorylated Ser129 α-synuclein antibody (phospho-syn) in the anterior cingulate cortex from a Parkinson's disease case. All sections were counterstained with Cresyl violet (blue) to visualize cell nuclei and membranes. The same adjustments to brightness were applied to all images. Scale bar represents 20 μm (applies to all panels).

In the Multiple system atrophy case (FIG. 15) images show glial-cell inclusions immunoreactive for our mAbs and commercially available mouse monoclonal anti-α-synuclein antibody (Syn-1) or mouse monoclonal anti-phosphorylated Ser129 α-synuclein antibody (phospho-syn) in the putamen from an MSA case. Sections were counterstained with Cresyl violet (blue) to visualise cell nuclei and membranes. The same adjustments to brightness were applied to all images. Scale bar represents 20 μm (applies to all panels).

### Example 11

Brain tissues samples from patients known to have neurodegenerative disorders were tested with the Syn-O2 antibody. The antibody was used at a 1 in 5000 (0.2 μg/ml) dilution. The antibodies produced intense staining in all patient samples.

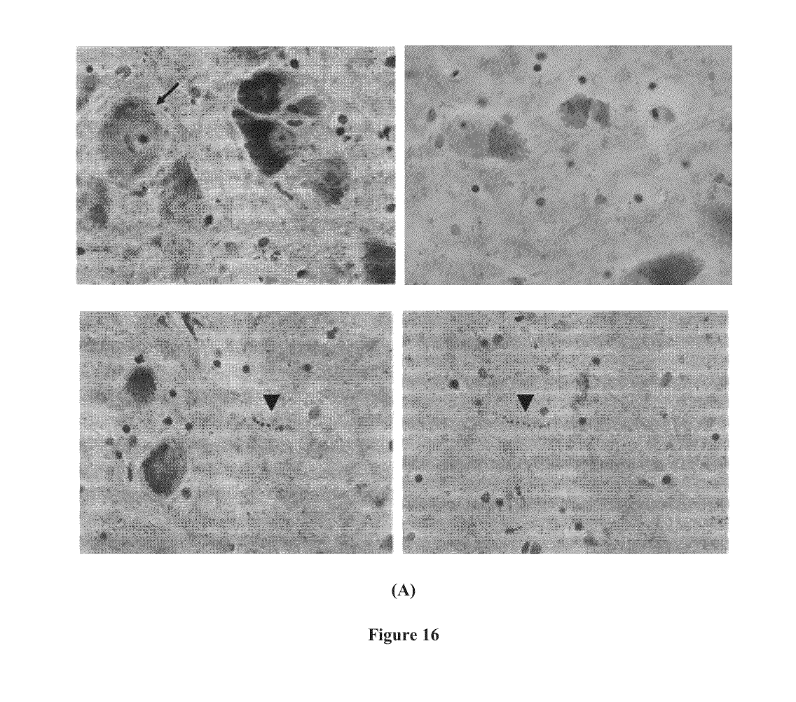

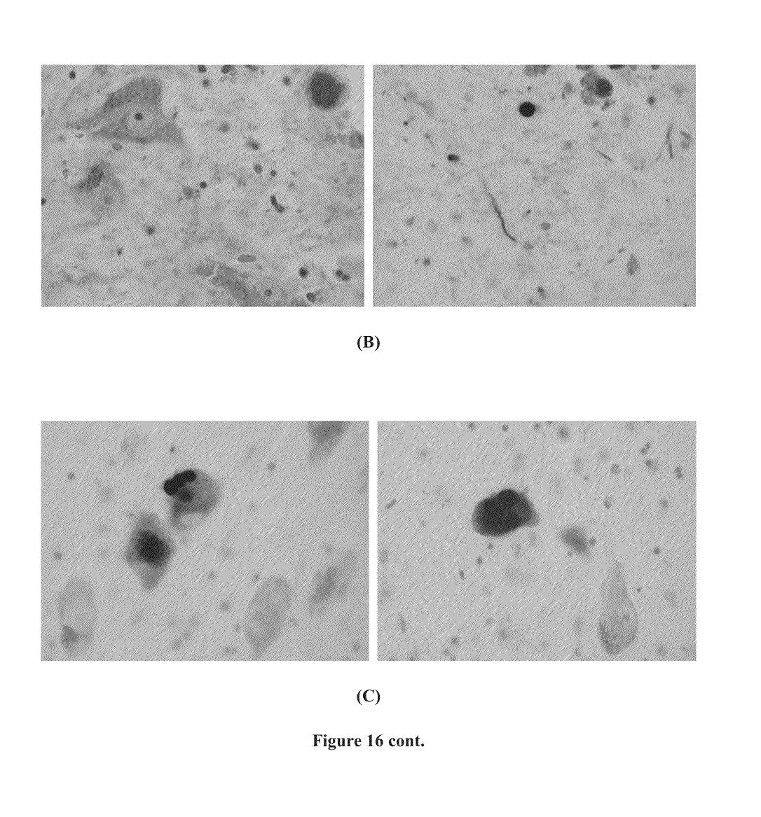

Patient A was known to have autosomal recessive early-onset Parkinsonism with heterozygous missense mutation in combination with heterozygous exon deletion in parkin gene. As shown in FIG. 16(A) oligomeric α-synuclein was observed in synapses and cellular processes (arrowheads). Very small aggregates were visible within the cytoplasm of the few neuromelanin-containing neurons (arrow).

Patient B was aprogressive DLB patent. As shown in FIG. 16(B) oligomeric α-synuclein immunoreactivity was observed in synapses, Lewy neurite-like structures and extracellular aggregates

Patient C has late-onset idiopathic Parkinson. As shown in FIG. 16(C) oligomeric α-synuclein immunoreactivity was observed in Lewy body-like intracytoplasmic inclusions and Lewy neurites.

These results shows that antibodies of the invention can be used to diagnosis neuroedegenerative diseases associated with α-synuclein.

### Example 12

Different sections of brain tissues samples (CA2 region of hippocampus, Entorhinal cortex superficial layers 1-3, Entorhinal cortex superficial layers 4-6, Entorhinal cortex neutries) were tested with the Syn-O1, Syn-O2, Syn-03 Syn-O4, Syn-F1 and Syn-F2 antibodies. The antibodies were used at a 1 in 5000 dilution. The brain samples went under 1 of four treatments 1) no pretreatment, 2) autoclaving at 120° C. for 10 minutes in citrate buffer, 3) formic acid for 15 mins or 4) 20 μg/ml proteinase K treatment.

Incubation was overnight at 4° C. Detection was with Histostain HP kit with Romulin as Chromogen. A control antibody, Syn-1 was used at a dilution of 1:1000. The tissues samples teasted with the control antibody were pretreated with Formic acid for 15 mins.

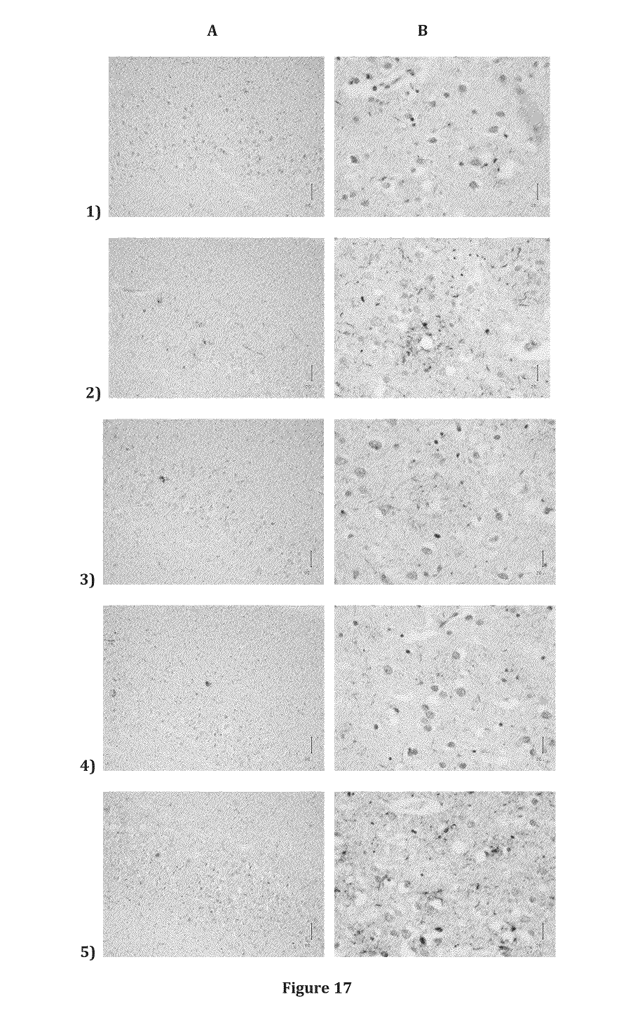

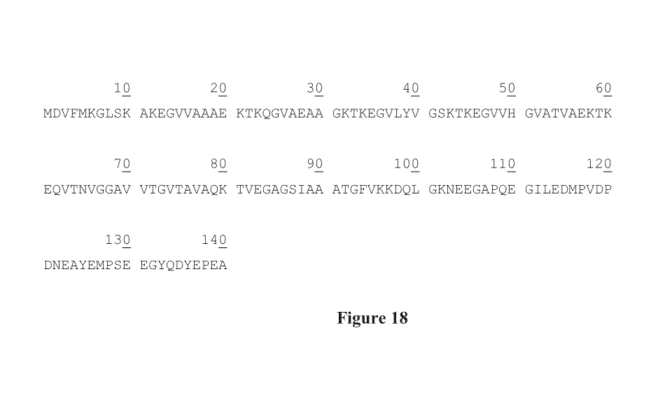

The results showed that the antibodies worked even without pretreatment of the brain tissues. However the antibodies are picked up better with pretreatment. Protein K treated samples showed the better results. Proteinase K treatment is known to enhance the immunoreactivity of abnormal synuclein (i.e. intracytoplasmic aggregates) and to decrease diffuse synaptic staining. The results for the control antibody and the syn-F2 antibody in the CA2 region of hippocampus (A), and the Entorhinal cortex neutries (B) are shown in FIG. 17.

The specific examples and embodiments described herein are exemplary only in nature and are not intending to be limiting of the invention. Further embodiments and examples, and advantages thereof, will be apparent to one of ordinary skill in the art in view of this specification and are within the scope of the claimed invention.

1. An antibody or fragment thereof having high affinity for α-synuclein aggregates and low affinity binding for synuclein monomers.

2. An antibody or fragment thereof according to claim 1 wherein the α-synuclein aggregates comprise protofibrils and/or soluble oligomers of α-synuclein.

3. An antibody or fragment thereof according to claim 1 wherein the α-synuclein aggregates comprise α-synuclein fibrils.

4. An antibody or fragment thereof according to claim 1 wherein the antibody or fragment thereof has a dissociation constant, Kd, of less than 10⁻⁷ M for human α-synuclein aggregates.

5. An antibody or fragment thereof according to claim 1 wherein the antibody or fragment thereof has a dissociation constant, Kd, of more than 10⁻⁵ M for monomeric α-synuclein.

6. Antibody or fragment thereof according to claim 1 wherein the antibody has a higher affinity for α-synucleins fibrils than for oligomeric forms of α-synucleins.

7. An antibody or fragment thereof according to claim 1 wherein the antibody or fragment thereof binds to an epitope comprising a C-terminal region of α-synuclein.

8. An antibody or fragment thereof according to claim 1 wherein the antibody or fragment thereof is a confirmational antibody.

9. An antibody or fragment thereof according to claim 1 wherein the antibody or fragment thereof do not recognise a linear epitope of α-synuclein.

10. An antibody or fragment thereof according to claim 1 wherein the antibody is a monoclonal antibody.

11. A pharmaceutical composition comprising an antibody or fragment according to claim 1 and a pharmaceutically acceptable diluent or carrier.

12. An antibody or fragment according to claim 1 for use as a medicament.

13. A method for preventing or treating a neurodegenerative disorder with α-synuclein pathology in an individual comprising administration of an antibody or fragment according to claim 1 to the individual.

14. A method according to claim 13, wherein the neurodegenerative disorder is Parkinson's disease, dementia with Lewy Bodies, Alzheimer's disease, multiple system atrophy, psychosis, schizophrenia or Creutzfeldt-Jakob disease.

15. A test kit for use in a method of determining whether or not an individual has a neurodegenerative disease comprising an antibody or fragment thereof according to claim 1.

16. A method of detecting α-synuclein fibrils and aggregates comprising the steps of
adding the antibody or fragment according to claim 1 to a biological sample, and
detecting the presence of a complex formed between α-synuclein fibrils and/or aggregates and the antibody or fragment.

17. A method for diagnosing a neurodegenerative disease associated with α-synuclein comprising:
adding the antibody or fragment according to claim 1 to a biological sample from a subject, and
detecting the presence or absence of a complex formed between α-synuclein aggregates and the antibody or fragment.

18. An antibody or fragment according to claim 1 for use as an imaging agent.

19. A method for imaging α-synuclein aggregates comprising
administering the antibody or fragment according to claim 1 to an individual, and
detecting the antibody.

20. The method according to claim 19 wherein the antibody comprises a detectable label.
